(19) United States
(12) Patent Application Publication
(10) Pub. No.: US 2019/0307886 A1
(43) Pub. Date: Oct. 10, 2019

(54) A CO-AMORPHOUS FORM OF A SUBSTANCE AND A PROTEIN

(71) Applicant: University of Copenhagen, Copenhagen K (DK)

(72) Inventors: Jaya Mishra, Copenhagen S (DK); Adam Bohr, Frederiksberg (DK); Thilo Berg, Lübeck (DE); Korbinian Löbmann, Copenhagen N (DK); Thomas Rades, Copenhagen N (DK); Holger Grohganz, Søborg (DK); Jorrit Water, Ferderiksberg (DK)

(21) Appl. No.: 16/469,593

(22) PCT Filed: Dec. 22, 2017
(86) PCT No.: PCT/DK2017/050449

§ 371 Date: Jun. 13, 2019

(30) Foreign Application Priority Data
Dec. 23, 2016 (DK) PA 2016 71043
Jul. 21, 2017 (DK) PA 2017 70586

(51) Int. Cl. A61K 47/42; A61K 47/34; A61K 9/20

(52) U.S. Cl. CPC A61K 47/42; A61K 9/2095; A61K 9/2063; A61K 47/34

(57) ABSTRACT

The present invention relates to co-amorphous form of a substance and a protein. The present invention also relates to pharmaceutical, cosmetic or veterinary compositions comprising the co-amorphous form as well as to methods for preparing and using the co-amorphous form.

### FIELD OF THE INVENTION

The present invention relates to co-amorphous forms of a substance and a protein. The present invention also relates to compositions such as pharmaceutical, cosmetic, veterinary, food or dietary compositions comprising the co-amorphous form as well as to methods for preparing and using the co-amorphous form.

### BACKGROUND OF THE INVENTION

Oral delivery is the preferred way of drug administration, since oral formulations are cheap to produce and convenient for the patient. However, oral formulation of crystalline drug substances with poor aqueous solubility is a major challenge for the pharmaceutical industry, since these substances exhibit poor solubility and low dissolution rates, resulting in low bioavailability and poor therapeutic performance.

Amorphous formulations have previously been used for addressing these issues. By converting the crystalline form of a drug into its amorphous counterpart, the solubility and dissolution rate of the drug substance is increased, leading to improved bioavailability and therapeutic efficacy (Hancock et al., Pharm. Res. 17 (2000) pp. 397-404). However, amorphous drug forms are physically unstable and tend to re-crystallize back into the poorly soluble crystalline form during storage (Laitinen et al., Int. J. Pharm. 453 (2013) pp. 65-79). Thus, methods for stabilizing amorphous drug forms are warranted by the pharmaceutical industry. Notably, there is a need in the art for new excipients that can further improve the stability and/or solubility properties of co-amorphous formulations.

### DETAILED DESCRIPTION OF THE INVENTION

The present invention is based on the surprising finding that when a protein or peptide, notably a native peptide or native protein, is used to produce a co-amorphous form of a poorly soluble substance such as a poorly soluble drug substance, the resulting co-amorphous form is a completely homogeneous, one-phase system in which the substance and the protein are combined at the molecular level. In this way, the aqueous solubility and the oral absorption is improved compared to an amorphous form of the substance itself without any protein excipient. Moreover, the physical stability of the co-amorphous form is also increased compared to the amorphous form of the drug itself and the physical mixture of the substance and the protein. Another advantage of the invention is that it may utilize inexpensive proteins or protein mixtures, which are produced in abundance as by-products during food production such as dairy production. Thus, the present invention provides a co-amorphous form of a substance and a protein. However, focus in the present context is on drug substances.

Amorphous forms of a drug substance as well as solid dispersions of a drug substance are known. Compared to such forms, a co-amorphous form of a drug substance and a protein has improved solubility and stability characteristics. When the co-amorphous form is for medical or cosmetic use, the protein should be physiologically acceptable and without any harmful pharmacological effect.

In an aspect, the present invention provides a co-amorphous form of a substance and a protein, wherein the protein is selected from whey protein isolate, whey protein hydrolysate, soy protein isolate, soy protein hydrolysate, glycinin, beta-conglycinin, legumin, vicilin, myoglobin, lysozyme, bovine serum albumin, egg white protein isolate, egg white protein hydrolysate, egg protein isolate, ovalbumin, ovomucin, ovoglobulin, avidin, ovomucoid, ovotransferrin, casein, alpha-lactalbumin, beta-lactoglobulin, immunoglobulin G, lactoferrin, keratin, rice protein isolate, rice protein hydrolysate, lentil protein isolate, pea protein isolate, faba bean protein isolate, chickpea protein isolate, cricket protein, silkworm protein, pumpkin seed protein, hemp protein, collagen, and gelatin.

The following proteins have been used in co-amorphous forms in the appended examples: proteins are whey protein isolate, whey protein hydrolysate, soy protein isolate, soy protein hydrolysate, myoglobin, lysozyme, egg protein isolate, egg white protein isolate, egg white protein hydrolysate, egg protein isolate, ovalbumin, casein, alpha-lactalbumin, beta-lactoglobulin, immunoglobulin G, rice protein isolate, rice protein hydrolysate, and collagen.

The hydrolysates are typically purchased. They may be prepared by exposing the protein isolate to high heat and a mixture of enzymes to denature and digest the protein into small fragments of a few amino acids. The whey protein hydrolysate supplier states on their webpage that they use enzymes to digest the proteins into smaller fragments assuming that they use a mixture of enzymes for this purpose.

In the examples herein, only used two individual proteolytic enzymes were used, trypsin and pepsin. These enzymes are present in the human GI tract to study if digestion by such individual enzymes would have an effect on the performance of the resulting product. In this way it is easier to identify the effect of hydrolysis based on the chains that are cleaved by the given enzyme. It is more difficult to identify individual effects of digestion by several enzymes used together like for the purchased WPH product.

It is natural that there are differences between the purchased WPH and the pepsin and trypsin digested whey proteins that the present inventors have prepared. The supplier of the hydrolysates most likely has optimized the enzyme mixture and ratios to obtain a safe and well performing product for people using these as sports supplements.

In order to select the best combination of protein and a substance, notably a drug substance, the following general observations were made (see the experimental section):

The highest increase in dissolution rate of a co-amorphous form of a protein and an active (drug) substance compared with the dissolution rate of the crystalline form of the active substance is seen when

i) the protein and drug molecule have opposite charge; the effects observed are most likely based on the charge of the substances allowing either attractive or repulsive forces. For the protein this is its net charge; or

ii) a protein is chosen, which contains a mixture of proteins; such proteins are whey protein isolate, rice protein isolate, egg protein isolate and soy protein isolate, or

iii) a high molecular weight protein is chosen, or

iv) whey protein isolate is chosen.

Regarding whey protein isolate, the examples herein show that whey protein isolate generally out-performs all other proteins tested and that the general guidelines mentioned above, not necessarily are valid for whey protein as it seems to have very suitable properties.

Regarding physical stability of the co-amorphous form (see Example 6), the examples herein show that co-amorphous form containing proteins like whey protein isolate, whey protein hydrolysate in general have excellent stability. It is interesting to note that co-amorphous forms based on whey protein isolate or whey protein hydrolysate have significantly improved physical stability compared with any other of the proteins tested. Co-amorphous forms containing proteins like ovalbumin, casein, collagen, lysozyme, myoglobin may have suitable stability dependent on the drug substance used. Co-amorphous forms with bovine serum albumin or with gelatin does not seem to have suitable stability.

As demonstrated in the examples, whey protein isolate and whey protein hydrolysate have proved to be suitable proteins for use in the present context. As these proteins contain a mixture of individual proteins/peptides/amino acids, it is contemplated that any combination of these proteins are suitable for use. Thus, especially relevant in connection with the present invention are proteins selected from beta-lactoglobulin, alpha-lactalbumin, immunoglobulin G, bovine serum albumin, and lactoferrin, and hydrolysates thereof. Bovine serum albumin should be present in such a combination in at the most 15% w/w such as at the most 10% w/w based on the total weight of the protein.

Notably, the present invention provides a co-amorphous form of a drug substance and a protein, wherein the protein is whey protein isolate or whey protein hydrolysate. Especially native whey protein isolate (non-denatured) is of interest. Whey protein isolate normally comprises beta-lactoglobulin, alpha-lactalbumin, immunoglobulin G, bovine serum albumin, and lactoferrin. However, whey protein isolate may also comprise other constituents such as (but not limited to) other proteins, peptides, carbohydrates, lipids minerals, vitamins and/or water to a smaller extent (in total not exceeding 5% w/w, such as from about 0 to about 5% w/w).

Whey protein isolate normally comprises from about 50 to about 70% of beta-lactoglobulin, from about 10 to about 25% of alpha-lactalbumin, from about 10 to about 20% of immunoglobulin G, about 1 to about 10% of bovine serum albumin, and about 1 to about 10% of lactoferrin.

Thus, proteins or protein mixtures containing
i) At least about 50% w/w of beta-lactoglobulin,
ii) At least about 10% w/w of alpha-lactalbumin,
iii) At least about 10% w/w of immunoglobulin G,
iv) At least about 1% w/w of bovine serum albumin, or
v) At least about 1% w/w of lactoferrin, or
vi) Mixtures thereof
   are within the scope of the present invention.

Although the constitution of commercially available whey protein isolate is well-defined, it cannot be ruled out that certain variations in content may occur. Accordingly, within the scope of the present invention are whey protein isolates as defined in the following:
i) Whey protein isolate containing:
   50-70% betalactoglobulin
   10-25% alpha-lactalbumin
   10-20% immunoglobulin G
   1-10% bovine serum albumin
   1-10% lactoferrin
ii) Whey protein isolate containing:
   50-70% betalactoglobulin
   10-25% alpha-lactalbumin
   10-15% immunoglobulin G
   5-10% bovine serum albumin
   1-5% lactoferrin
iii) Whey protein isolate containing:
   52-72% betalactoglobulin
   14-22% alpha-lactalbumin
   11-16% immunoglobulin G
   2-8% bovine serum albumin
   2-8% lactoferrin
iv) Whey protein isolate containing:
   53-68% betalactoglobulin
   14-22% alpha-lactalbumin
   11-15% immunoglobulin G
   4-8% bovine serum albumin
   2-6% lactoferrin

Commercially available whey protein isolates are described as comprising from about 55 to about 65% of beta-lactoglobulin, from about 15 to about 21% of alpha-lactalbumin, from about 13 to about 14% of immunoglobulin G, about 7% of bovine serum albumin, and about 3% of lactoferrin (De Wt, Journal of Dairy Science 81 (1998) pp. 597-608 and Jenness, Protein Composition of Milk in Milk Proteins V1: Chemistry and Molecular Biology, Academic Press, 2012). To be more specific, whey protein isolates for use according to the invention include:
i) Whey protein isolate containing:
   55% betalactoglobulin,
   21% alpha-lactalbumin,
   14% immunoglobulin G,
   7% bovine serum albumin, and
   3% lactoferrin.
ii) Whey protein isolate containing:
   65% betalactoglobulin,
   15% alpha-lactalbumin,
   13% immunoglobulin G,
   7% bovine serum albumin, and
   0% lactoferrin.

As mentioned above, another suitable whey protein for use in a co-amorphous form according to the invention is whey protein hydrolysate. Whey protein hydrolysate is a mixture of proteins/peptides/amino acids derived from whey protein isolate that has been subjected to any form of chemical, enzymatic, physical, or mechanical degradation and optionally purified to yield the hydrolysate comprising the corresponding degradation products of whey protein isolate.

The present invention has particular interest for substances that have a low aqueous solubility and where an increase in aqueous solubility or dissolution rate is desired. The invention is also of interest in those cases, where a substance preferably is used in amorphous form, but where the amorphous form does not have a suitable storage stability. Such substances include catalysts, chemical reagents, nutrients, food ingredients, enzymes, bactericides, pesticides, fungicides, disinfectants, fragrances, flavours, fertilizers, and micronutrients as well as drug substances.

The main focus of the present invention is when the substance is a drug substance that is therapeutically, prophylactically, and/or diagnostically active. Alternatively, the substance may be useful for therapeutic, prophylactic, or diagnostic purposes.

In the present context, a low solubility of a drug substance is defined according to the Biopharmaceutics Classification System (BCS) as provided and defined by the US Food and Drug Administration (FDA). The term “solubility” refers herein to the ability of a compound to dissolve in a solvent to form a solution. Particularly relevant for the present disclosure is the definition of the terms ‘poorly soluble or insoluble’ according to the four different classes of drugs:
Class I—High Permeability, High Solubility (neither permeability nor solubility limits the oral bioavailability of the drug compound)
Class II—High Permeability, Low Solubility (low solubility limits the oral bioavailability of the drug compound)
Class III—Low Permeability, High Solubility (low permeability limits the oral bioavailability of the drug compound)
Class IV—Low Permeability, Low Solubility (both permeability and solubility limit the oral bioavailability of the drug compound)

According to this classification, a drug substance has low solubility if the highest dose strength is not soluble in 250 ml of aqueous medium or less over a pH range of 1 to 7.5.

A solvent for use in determining the solubility of a substance is an aqueous medium. The aqueous medium may contain one or more pH adjusting agents or buffering agents to ensure a specific pH in the range of from 1 to 7.5, or it may be water.

Of interest is a co-amorphous form according to the invention that contains drug substance that normally cannot be administered by the oral route such as BCS class 4 drugs. Other drug substances of interest may be those that cannot be administered orally e.g. due to presence of an efflux pump or similar physiological mechanisms that decrease or prevent uptake of the drug substance. For such drug substances, a markedly improved formulation is desired in order to avoid administration solely by the parenteral route, which normally involves educated health care personnel.

However, it is contemplated that the present concept is of a general character, i.e. it can be applied to all types of drug substances for which an improved stability of solubility is advantageous. Such drug substance may be selected from antibiotics such as amoxicillin, anti-infective agents such as acyclovir, albendazole, anidulafungin, azithromycin, cefdinir, cefditoren, cefixime, cefotiam, cefpodoxime, cefuroxime axetil, chlarithromycin, chloroquine, ciprofloxacin, clarithromycin, clofazimine, cobicistat, dapsone, daptomycin, diloxanide, doxycycline, efavirenz, elvitegravir, erythromycin, etravirine, griseofulvin, indinavir, itraconazole, ivermectin, linezolid, lopinavir, mebendazole, mefloquine, metronidazole, mycamine, nalidixic acid, nelfinavir, nevirapine, niclosamide, nitrofurantoin, nystatin, praziquantel, pyrantel, pyrimethamine, quinine, rifampicin, rilpivirine, ritonavir, roxithromycin, saquinavir, sulfadiazine, sulfamethoxazole, sultamicillin, tosufloxacin, and trimethoprim,

antineoplastic agents such as bicalutamide, cyproterone, docetaxel, gefitinib, imatinib, irinotecan, paclitaxel, and tamoxifen, cardiovascular agents such as acetazolamide, atorvastatin, azetacolamide, benidipine, candesartan, cilexetil, carvedilol, cilostazol, clopidogrel, eprosartan, ethyl icosapentate, ezetimibe, fenofibrate, furosemide, hydrochlorothiazide, irbesartan, lovastatin, manidipine, nifedipine, nilvadipine, olmesartan, simvastatin, spironolactone, telmisartan, ticlopidine, triflusal, valsartan, verapamil, and warfarin,

CNS agents such as aceclofenac, acetaminophen, acetylsalicylic acid, apriprazole, carbamazepine, carisoprodol, celecoxib, chlorpromazine, clonazepam, clozapine, diazepam, diclofenac, flurbiprofen, haloperidol, ibuprofen, ketoprofen, lamotrigine, levodopa, lorazepam, meloxicam, metaxalone, methylphenidate, metoclopramide, modafinil, nabilone, nabumetone, nicergoline, nimesulide, olanzapine, oxcarbazepine, oxycodone, phenobarbital, phenytoin, quetiapine, risperidone, rofecoxib, sertraline, sulpiride, valproic acid, and zlatoprofen,

dermatological agents such as isotretinoin, endocrine and metabolic agents such as cabergoline, dexamethasone, epalrestat, estrone sulphate, glibenclamide, gliclazide, glimpiride, glipizide, medroxyprogesterone, norethindrone acetate, pioglitazone, prednisone, propylthiouracil, and raloxifene,

gastrointestinal agents such as bisacodyl, famotidine, mesalamie, mosapride, orlistat, rebamipide, sennoside A, sulfasalazine, teprenone, and ursodeoxycholic acid,

nutritional agents such as folic acid, menatetrenone, retinol, and tocopherol nicotinate,

respiratory agents such as ebastine, hydroxyzine, L-carbocysteine, loratadine, pranlukast, and theophylline,

anti-hyperuricemic agents such as allopurinol,

and agents for treating erectile dysfunction such as sildenafil and tadalafil.

In relation to the above-mentioned drug substances it is contemplated that co-amorphous forms of any of these drug substances and a protein selected those mentioned herein, notably from whey protein isolate or whey protein hydrolysate or from its consituents or any combination thereof (i.e. alpha-lactalbumin, beta-lactoglobulin, immunoglobulin G, bovine serum albumin, and lactoferrin) will provide a benefit in terms of improved pharmaceutical properties such as improved stability and solubility. Notably, the protein is whey protein isolate and/or whey protein hydrolysate.

A co-amorphous form of a substance and a protein according to the invention may contain from 1-95% w/w of the substance and from 5 to 99% w/w of the protein. Thus, co-amorphous forms may contain from about 2.5 to 90% w/w or from about 10 to about 80% w/w of the substance. As seen from the examples, the desired results can be obtained with various concentrations of the drug substance and the protein. Suitable examples include co-amorphous forms containing from about 25 to about 75% w/w of a drug substance.

A co-amorphous form according to the invention may be formulated into a suitable application form dependent on the specific use of the form. In those cases where the substance is for medical or cosmetic use the co-amorphous form may be formulated into pharmaceutical or cosmetic compositions. Such compositions include compositions for oral, topical, mucosal, pulmonary, parenteral, sublingual, nasal, occular and enteral administration. The oral administration route is preferred, if possible.

Such compositions may include one or more pharmaceutically or cosmetically acceptable excipients. A person skilled in pharmaceutical or cosmetic formulation will know how to formulate specific compositions e.g. with guidance from Remington's Pharmaceutical Sciences, 18th edition, Mack Publishing Company, 1990.

Given a specific substance, a protein for forming a co-amorphous form may be selected based on the physicochemical properties of the individual components. Such a selection or matching could be performed according to size (in terms of e.g. molecular weight and/or hydrodynamic volume), hydrophobicity (e.g. hydrophobic substance/hydrophobic protein or hydrophilic substance/hydrophilic protein), and/or electrostatic interactions (e.g. anionic substance/cationic protein, cationic substance/anionic protein, and neutral substance/neutral protein) However, other criteria for selection and matching may also be envisioned depending on the substance in question.

In an aspect, the present invention provides a method for preparing a co-amorphous form of a substance and a protein, wherein the co-amorphous form is prepared by thermodynamic methods such as spray drying, solvent evaporation, freeze drying, precipitation from supercritical fluids, melt quenching, hot melt extrusion, 2D printing, and 3D printing, or by kinetic disordering processes such as any kind of milling process including ball milling and cryo-milling.

As appears from the examples herein, spray drying provides excellent results.

A method for preparing a co-amorphous form as defined by the invention comprises:
i) placing a substance and a protein in a container, and sealing the container,
ii) physically disordering the substance together with the protein by mechanical activation until the substance and the protein are completely disrupted resulting in a co-amorphous product,
iii) simultaneously mixing of the substance and the protein to obtain a homogeneous co-amorphous one-phase system comprising the substance and the protein.

Another method for preparing a co-amorphous form as defined by the invention comprises:
i) dissolving a substance and a protein in a solvent or solvent mixture to form a single phase solution,
ii) removing the solvent from the resulting solution from step i)
to obtain a homogeneous one-phase co-amorphous mixture comprising the substance and the protein.

Yet another method preparing a co-amorphous form as defined by the invention comprises:
i) dissolving a substance and a protein in a solvent or solvent mixture to form a single phase solution,
ii) freezing the single phase solution from step i),
iii) removing the solvent or solvent mixture through sublimation from the resulting frozen single phase from step ii)
to obtain a homogeneous one-phase co-amorphous mixture comprising the substance and the protein.

As seen from the examples herein suitable solvents are water of aqueous solutions. pH regulation does not seem to be necessary in order to obtain co-amorphous forms neither does organic solvents seem to be necessary. In the examples, water has been used as solvent.

Yet another method for preparing a co-amorphous form as defined in the invention comprises:
i) mixing a substance and a protein to obtain a physical mixture of both components,
ii) disordering the resulting physical mixture from step i) by heating the mixture above the melting point of either the drug, the protein or both together to obtain a homogeneous single phase melt comprising both the substance and the protein,
iii) cooling of the single phase melt from step ii) to below the glass transition temperature
to obtain a homogeneous one-phase co-amorphous mixture comprising the substance and the protein.
   Co-Amorphous Forms of a Drug Substance and a Protein where the Drug Substance is a Substrate to Efflux Pump(s) in the Gastrointestinal System

Of particular interest are co-amorphous forms of a protein and a drug substance such as anti-cancer drug substances that are normally administered by the oral route, but for which alternative formulations are wanted to improve therapeutic efficacy and patient compliance.

In order to have a therapeutic effect, any orally administered drug substance must first dissolve in the intestinal fluids and subsequently permeate the intestinal wall. Thus, sufficient aqueous dissolution and intestinal permeability of the drug substance are important to obtain acceptable bioavailability. However, many drug substances such as anti-cancer drug substances show poor aqueous solubility, resulting in a low oral bioavailability and thus inefficient drug action.

Another reason for poor bioavailability can also be poor intestinal absorption. Poor absorption of many drug substances such as some anti-cancer drugs results from such drug substances being substrate to so-called intestinal efflux pumps such as P-glycoprotein (also known as multidrug resistance protein or MDR1, which in addition to gastrointestinal tract also is located in the liver and kidneys and in the blood-brain barrier). Such efflux pumps are typically situated in the absorption cell layer of the intestine and their main purpose is to protect the body by repumping foreign or toxic substances back into the intestinal lumen. Many drug substances such as some anti-cancer drug substances are substrates to these efflux pumps. However, some anti-cancer drug substances such as bicalutamide also show efflux pump inhibition in addition to their anti-cancer effects.

For some drug substances such as the anti-cancer drug docetaxel, the situation becomes more challenging because said drug substances are both poorly soluble and poorly absorbable, resulting in two delivery barriers. For this reason, the preferred route of administration for these drug substances is via intravenous infusion. However, as the drug substances is very poorly soluble it is still necessary to add solubilizers and solvents, which may be harmful to the body and may cause irritation and severe allergic reactions. The injectable formulations further need to be sterile, which is costly and still holds the risk of infection. Moreover, trained staff is required for administration since patients need to be hospitalized for the duration of the infusion. Finally, intravenous therapies such as chemotherapies are generally less favourable than their oral counterparts as they are usually given once every 2-3 weeks, thus resulting in a less uniform plasma profile of the drug substance compared with the daily oral therapies. Thus, technologies that allow changing an intravenous therapy to an oral therapy carry many advantages.

Co-amorphous forms such as co-amorphous forms of a drug substance and a protein provide a method for oral administration of drug substances that are normally only available by the intravenous route, since co-amorphous forms increase the solubility and stability of the drug substance, resulting in increased bioavailability.

In particular, co-amorphous forms can be used to co-deliver a poorly soluble drug substance such as docetaxel that is a substrate for an efflux pump such as P-glycoprotein and another poorly soluble drug substance such as bicalutamide that in addition to its therapeutic effect is an inhibitor of said efflux pump. By including such drug substances in the same co-amorphous form, the drug substances may stabilize each other in the amorphous form via intermolecular interactions such as hydrogen bonding or ionic interactions. As a result of the stable amorphous system, both of the poorly soluble drug substances achieve a higher solubility and stability, which leads to a higher amount of dissolved drug substance in the gastrointestinal tract available for absorption. Moreover, by including an efflux pump substrate and an efflux pump inhibitor in the same co-amorphous form, the uptake of the efflux pump substrate will be improved, which results in increased oral bioavailability.

In addition to the pair of bicalutamide and docetaxel, the following pairs exemplify a combination of an efflux pump substrate and an efflux pump inhibitor:

talinolol and naringin, and ritonavir and quercetin.

Other examples can be found in the literature and are within the scope of the present invention where one or more drug substance(s) have been co-amorphized with a protein.

### Definitions

### “Substance”:

According to the present invention, the term “substance” in the context of co-amorphous forms is defined as one or more substances. Thus, according to the present invention, the term “co-amorphous form of a substance and a protein” describes co-amorphous forms comprising one or more substances. The term “drug substance” describes a therapeutically or prophylactically active substance.

### “Protein”:

According to the present invention, the term “protein” used in the context of co-amorphous forms relates to one or more proteins such as single proteins, protein mixtures, protein/peptide/amino acid mixtures, protein/amino acid mixtures, and peptide/amino acid mixtures

“Whey protein isolate”:

According to the present invention, whey protein isolate (WPI) is defined as a mixture of proteins comprising beta-lactoglobulin, alpha-lactalbumin, immunoglobulin G, bovine serum albumin, and/or lactoferrin.

Normally, whey protein isolate comprises from about 50 to about 70% w/w of beta-lactoglobulin, from about 10 to about 25% w/w of alpha-lactalbumin, from about 10 to about 20% w/w of immunoglobulin G, about 1 to about 10% w/w of bovine serum albumin, and about 1 to about 10% w/w of lactoferrin. Optionally, whey protein isolate may also comprise other constituents such as (but not limited to) other proteins, peptides, carbohydrates, lipids, minerals, vitamins or water to a smaller extent (in total not exceeding 5% w/w).

### “Whey Protein Hydrolysate”

According to the present invention, whey protein hydrolysate is defined as a mixture of proteins/peptides/amino acids derived from whey protein isolate that has been subjected to any form of chemical, enzymatic, physical, or mechanical degradation process and optionally purified to yield the hydrolysate comprising the corresponding degradation products of the whey protein isolate.

### “Co-Amorphous”:

According to the present invention, the term “co-amorphous” refers to a combination of two or more components that form a homogeneous amorphous one-phase system where the components are intimately mixed on the molecular level. The “co-amorphous” samples can be prepared by thermodynamic methods, or by kinetic disordering processes. XRPD, together with DSC, can be used to identify whether the sample is “co-amorphous” after preparation.

### LEGENDS TO FIGURES

FIG. 1

XRPD diffractograms of co-amorphous forms of indomethacin (IND), carvedilol (CAR), paracetamol (PAR) and furosemide (FUR) with either whey protein isolate (WPI) or whey protein hydrolysate (WPH). All co-amorphous forms were obtained by spray drying. Panel (i): A=IND-WPI, B=CAR-WPI, C=PAR-WPI, D=FUR-WPI. Panel (ii): A=IND-WPH, B=CAR-WPH, C=PAR-WPH, D=FUR-WPH.

FIG. 2

Intrinsic dissolution rate of crystalline indomethacin (C IND), amorphous indomethacin (A IND), co-amorphous indomethacin-whey protein isolate obtained by ball milling (BM IND-WPI), co-amorphous indomethacin-whey protein hydrolysate obtained by ball milling (BM IND-WPH), co-amorphous indomethacin-whey protein isolate obtained by spray drying (SD IND-WPI), and co-amorphous indomethacin-whey protein hydrolysate obtained by spray drying (SD IND-WPH).

FIG. 3

Intrinsic dissolution rate of (a) crystalline carvedilol (C CAR), amorphous carvedilol (A CAR), co-amorphous carvedilol-whey protein isolate obtained by ball milling (BM CAR-WPI), co-amorphous carvedilol-whey protein hydrolysate obtained by ball milling (BM CAR-WPH), co-amorphous carvedilol-whey protein isolate obtained by spray drying (SD CAR-WPI), and co-amorphous carvedilol-whey protein hydrolysate obtained by spray drying (SD CAR-WPH); (b) crystalline paracetamol (C PAR), amorphous paracetamol (A PAR), co-amorphous paracetamol-whey protein isolate obtained by ball milling (BM PAR-WPI), co-amorphous paracetamol-whey protein hydrolysate obtained by ball milling (BM PAR-WPH), co-amorphous paracetamol-whey protein isolate obtained by spray drying (SD PAR-WPI), and co-amorphous paracetamol-whey protein hydrolysate obtained by spray drying (SD PAR-WPH); (c) crystalline furosemide (C FUR), amorphous furosemide (A FUR), co-amorphous furosemide-whey protein isolate obtained by ball milling (BM FUR-WPI), co-amorphous furosemide-whey protein hydrolysate obtained by ball milling (BM FUR-WPH), co-amorphous furosemide-whey protein isolate obtained by spray drying (SD FUR-WPI), and co-amorphous furosemide-whey protein hydrolysate obtained by spray drying (SD FUR-WPH)

FIG. 4

Intrinsic dissolution rate of (a) crystalline indomethacin (C IND), amorphous indomethacin (A IND), co-amorphous indomethacin-whey protein isolate obtained by spray drying (SD IND-WPI), co-amorphous indomethacin with whey protein isolate (digested with trypsin) obtained by spray drying (SD IND-WPI ENZ T), co-amorphous indomethacin with whey protein isolate (digested with trypsin followed by pepsin) obtained by spray drying (SD IND-WPI ENZ T+P), co-amorphous indomethacin with whey protein isolate (digested with pepsin) obtained by spray drying (SD IND-WPI ENZ P), co-amorphous indomethacin with whey protein isolate (digested with pepsin followed by trypsin) obtained by spray drying (SD IND-WPI ENZ P+T); (b) crystalline indomethacin (C IND), amorphous indomethacin (A IND), co-amorphous indomethacin-whey protein isolate obtained by spray drying (SD IND-WPI), co-amorphous indomethacin-bovine serum albumin obtained by spray drying (SD IND-BSA), co-amorphous indomethacin-alpha-lactalbumin obtained by spray drying (SD IND-a lactalbumin), and co-amorphous indomethacin-beta-lactoglobulin obtained by spray drying (SD IND-b lactoglobulin).

FIG. 5

Powder dissolution studies of crystalline indomethacin (C IND), amorphous indomethacin (A IND), physical mixture of indomethacin and whey protein isolate (PM IND-WPI), co-amorphous form of indomethacin and whey protein isolate obtained by ball milling (BM IND-WPI), and co-amorphous form of indomethacin and whey protein isolate obtained by spray drying (SD IND-WPI).

FIG. 6

Stability of co-amorphous forms of whey protein isolate (WPI) with indomethacin (IND), carvedilol (CAR), paracetamol (PAR) and furosemide (FUR), respectively. The 5 months stability data was measured for WPI mixtures with IND, CAR and FUR and the 1 month stability was measured for PAR-WPI, assessed using x-ray powder diffraction (XRPD). All co-amorphous mixtures were obtained by spray drying. A=PAR-WPI, B=FUR-WPI, C=CAR-WPI, D=IND-WPI. Stability studies were further carried out each month until the drug substance started to recrystallize and the data is shown in Table 3.

FIG. 7

Absolute bioavailability of crystalline furosemide (Crystalline FUR), amorphous furosemide (Amorphous FUR), co-amorphous furosemide-polyvinylpyrrolidone (25:75 w/w) obtained by spray drying (SD FUR-PVP (75:25)), physical mixture (50:50 w/w) of furosemide-whey protein isolate and (PM FUR-WPI (50:50)), co-amorphous furosemide-whey protein isolate (25:75 w/w) obtained by spray drying (SD FUR-WPI (25:75)), co-amorphous furosemide-whey protein isolate (50:50 w/w) obtained by spray drying (SD FUR-WPI (50:50)), and co-amorphous furosemide-whey protein isolate (75:25 w/w) obtained by spray drying (SD FUR-WPI (75:25)). Bioavailability was assessed following oral administration to rats. Polyvinylpyrrolidone was included in the experiment because it is the most commonly used excipient for making solid dispersions, which is the main competing technology for amorphization in terms of optimizing solubility and/or stability of drug substances with poor solubility and/or stability properties. The ratios of WPI and FUR were varied by changing the content of WPI while the content of FUR was kept constant.

FIG. 8

Maximum concentration in the bloodstream (Cmax) of crystalline furosemide (Crystalline FUR), amorphous furosemide (Amorphous FUR), co-amorphous furosemide-polyvinylpyrrolidone (25:75 w/w) obtained by spray drying (SD FUR-PVP (75:25)), physical mixture (50:50 w/w) of furosemide-whey protein isolate and (PM FUR-WPI (50:50)), co-amorphous furosemide-whey protein isolate (25:75 w/w) obtained by spray drying (SD FUR-WPI (25:75)), co-amorphous furosemide-whey protein isolate (50:50 w/w) obtained by spray drying (SD FUR-WPI (50:50)), and co-amorphous furosemide-whey protein isolate (75:25 w/w) obtained by spray drying (SD FUR-WPI (75:25)). Cmax was assessed following oral administration to rats. Polyvinylpyrrolidone was included in the experiment because it is the most commonly used excipient for making solid dispersions, which is the main competing technology for amorphization in terms of optimizing solubility and/or stability of drug substances with poor solubility and/or stability properties. The ratios of WPI and FUR were varied by changing the content of WPI while the content of FUR was kept constant.

FIG. 9

Intrinsic dissolution rate of crystalline furosemide (Crystalline FUR), amorphous furosemide (Amorphous FUR), co-amorphous furosemide-polyvinylpyrrolidone (25:75 w/w) obtained by spray drying (SD FUR-PVP (75:25)), physical mixture (50:50 w/w) of furosemide-whey protein isolate and (PM FUR-WPI (50:50)), co-amorphous furosemide-whey protein isolate (25:75 w/w) obtained by spray drying (SD FUR-WPI (25:75)), co-amorphous furosemide-whey protein isolate (50:50 w/w) obtained by spray drying (SD FUR-WPI (50:50)), and co-amorphous furosemide-whey protein isolate (75:25 w/w) obtained by spray drying (SD FUR-WPI (75:25)). Polyvinylpyrrolidone was included in the experiment because it is the most commonly used excipient for making solid dispersions, which is the main competing technology for amorphization in terms of optimizing solubility and/or stability of drug substances with poor solubility and/or stability properties. The ratios of WPI and FUR are varied by changing the content of WPI while the content of FUR is kept constant.

FIG. 10

XRPD diffractograms of co-amorphous forms of indomethacin (IND) with various proteins. All co-amorphous forms were obtained by spray drying. A: SD IND-Soy, B: SD IND-Rice, C: SD IND-Egg, D: SD IND-Gelatin, E: SD IND-Collagen, F: SD IND-Myoglobin, G: SD IND-Lysozyme and H: SD IND-Casein.

FIG. 11 (i) and (ii)

Intrinsic dissolution rate of (i) SD IND-Gelatin, SD IND-Egg, SD IND-Soy, C IND, A IND; and (ii) SD IND-Myoglobin, SD IND-Lysozyme, SD IND-Collagen, SD IND-Casein, C IND, A IND.

FIG. 12

XRPD diffractograms of A: SD IND-Ovalbumin, B: SD CEL-WPI, C: SD CEL-Myoglobin, D: SD CEL-Lysozyme, E: SD CEL-Casein, F: SD CEL-Collagen.

FIG. 13

Intrinsic dissolution rate of (i) SD IND-Myoglobin, SD IND-Lysozyme, SD IND-Collagen, SD IND-Casein, SD IND-WPI; (ii) SD CEL-Myoglobin, SD CEL-Lysozyme, SD CEL-Collagen, SD CEL-Casein, SD CEL-WPI; and (iii) SD CAR-Myoglobin, SD CAR-Lysozyme, SD CAR-Collagen, SD CAR-Casein, SD CAR-WPI.

FIG. 14

Intrinsic dissolution rate of (i) SD IND-EGG, SD IND-RICE, SD IND-SOY, SD IND-WPI, SD IND-Gelatin; and (ii) SD IND-BSA, SD IND-Ovalbumin, SD IND-Casein, SD IND-WPI.

FIG. 15

Intrinsic dissolution rate (IDR) of (i) SD IND-Myoglobin, SD IND-Lysozyme, SD IND-Collagen, SD IND-Casein, SD IND-WPI; ii) SD CEL-Myoglobin, SD CEL-Lysozyme, SD CEL-Collagen, SD CEL-Casein, SD CEL-WPI; and iii) SD CAR-Myoglobin, SD CAR-Lysozyme, SD CAR-Collagen, SD CAR-Casein, SD CAR-WPI; Where the IDRs are plotted as a function of isoionic points (pI) of the proteins.

FIG. 16

Intrinsic dissolution rate (IDR) of SD IND-BSA, SD IND-Ovalbumin, SD IND-Casein, SD IND-WPI; where (i) the IDRs are plotted as a function of molecular weight (Mw) of the proteins; and (ii) the IDRs are depicted as a function of isoionic points (pI) of the proteins.

FIG. 17

Intrinsic dissolution rate (IDR) of SD IND-EGG, SD IND-RICE, SD IND-SOY, SD IND-WPI, SD IND-Gelatin; where the IDRs are depicted as a function of isoionic points (pI) of the proteins.

FIG. 18

XRPD diffractograms of A: SD CAR-Myoglobin, B: SD CAR-Lysozyme, C: SD CAR-Collagen, D: SD CAR-Casein.

FIG. 19

XRPD diffractograms of SD IND-WPI and SD IND-WPH, 20 months after preparation of respective co-amorphous formulations.

### ABBREVIATIONS

BM, ball milling
CAR, carvedilol
CEL, celecoxib
FUR, furosemide
IDR, intrinsic dissolution rate
IND, indomethacin
pI, isoionic point
LC-MS, liquid chromatography-mass spectometry
mDSC, modulated differential scanning calorimetry
Mw, molecular weight
PAR, paracetamol
PM, physical mixture
PVP, polyvinylpyrrolidone
SD, spray drying
TGA, thermogravimetric analysis
UV Vis, ultra-violet spectrophotometry
XRPD, x-ray powder diffraction
WPH, whey protein hydrolysate
WPI, whey protein isolate

### EXPERIMENTALS

### Materials

Indomethacin (IND) was purchased from Hawkins, Inc. (Minneapolis, Minn., USA). Carvedilol (CAR) from Cipla Ltd. (Mumbai, India), paracetamol (PAR) from Fagron (Copenhagen, Denmark) and Furosemide (FUR) from Sigma-Aldrich (St. Louis, Mo., USA). All these powders were of reagent grade and used as received. Whey protein isolate (WPI), whey protein hydrolysate (WPH), rice protein isolate, soy protein isolate and egg protein isolate were purchased from LSP Sporternahrung (Bonn, Germany, www.lsp-sports.de). Polyvinylpyrrolidone (PVP, Kollidon® 25), alpha-lactalbumin and beta-lactoglobulin from bovine milk were received from Sigma-Aldrich (Schnelldorf, Germany). Bovine serum albumin (BSA), celecoxib (CEL), ovalbumin, collagen, gelatin, myoglobin, lysozyme, casein and, pepsin from porcine gastric mucosa and trypsin from bovine pancreas were obtained from Sigma-Aldrich (Brøndby, Denmark). All materials were of reagent grade and used as received.

### Methods

### Spray Drying:

Physical mixtures (powders mixed together in container with spatula) of IND, CAR, PAR, CEL and FUR with either WPI or WPH were prepared at a 1:1 weight ratios. The mixtures were then dissolved in 250 ml of milliQ water (18.2 MΩ, 23.8° C.) freshly prepared by MilliQ water system from LabWater (Los Angeles, Calif., USA). The concentration of drug substance-WPI/WPH in each respective solution was 4 mg/ml. Spray drying was performed using a Büchi B-290 spray-dryer (Büchi Labortechnik AG, Flawil, Switzerland) equipped with a dehumidifier (Büchi B296). The spray drying conditions were as follows: inlet temperature: 120° C.; outlet temperature: approx. 62° C.; atomizing air flow rate: 667 l/h; drying air flow (nitrogen): 40 m³/h and feed flow rate: 9 ml/min. To further study the dissolution behaviour of the co-amorphous forms, IND was spray dried together with the main components of WPI (alpha-lactalbumin, beta-lactoglobulin and bovine serum albumin (BSA), respectively), and with WPI subjected to enzymatic digestion (trypsin, pepsin, trypsin followed by pepsin, and pepsin followed by trypsin, respectively). Enzymatic digestions was performed overnight using 1 mg enzyme for every 100 mg WPI. Pepsin digestion was performed at pH 8 and trypsin digestion was performed at pH 3. To analyze the dissolution behaviour of a drug substance with proteins of different properties, IND was spray dried with rice protein isolate, egg protein isolate, soy protein isolate, ovalbumin, collagen, gelatin, myoglobin, lysozyme and casein, and CEL and CAR were spray dried with WPI, myoglobin, lysozyme, collagen and casein.

### Ball Milling:

To compare the co-amorphous forms obtained with spray drying to the co-amorphous forms obtained by ball milling, physical mixtures of IND, CAR, PAR, and FUR with either WPI or WPH were subjected to vibrational ball milling using a MixerMill MM400 (Retsch GmbH & Co., Haan, Germany) in a cold room (4° C.). The co-amorphous forms obtained by ball milling were produced by placing a total mass of 700 mg of 1:1 weight ratios (drug substance-WPI/WPH) in 25 ml milling jars with two 12 mm stainless steel balls. Milling was performed at 30 Hz for up to 30 min in case of IND and CAR and up to 60 min in case of FUR and PAR.

### X-Ray Powder Diffraction (XRPD) for Measurement of Solid State Form:

The molecular interactions of the drug substance-WPI/WPH mixtures were investigated by XRPD using an X'Pert PANanalytical PRO X-ray diffractometer (PANanalytical, Almelo, The Netherlands) with Cukα radition: 1.54187 Å, current: 40 mA and acceleration voltage: 45 kV. Each of the co-amorphous forms obtained either by spray drying or ball milling were scanned (scan rate of 0.067° 2θ/s and step size of 0.026°) with reflectance mode between 2° and 35° 2θ. The collected data was analysed using X'Pert PANanalytical Collector software (PANanalytical, Almelo, The Netherlands).

Thermogravimetric analysis for measurement of residual moisture:

Thermogravimetric analysis was performed on a TGA Discovery instrument (TA Instruments, New Castle, USA). Samples of 10 mg were placed in a platinum pan and sealed with a lid and heated from 25 to 300° C. at 10° C./min. Resulting weight-temperature diagrams were analyzed using Trios software (TA Instruments, New Castle, USA) to calculate the weight loss between 25 and 150° C.

Modulated differential scanning calorimetry (mDSC) for measurement of Tg and Tm: Thermal analysis was performed using a Discovery DSC instrument (TA Instruments, New Castle, USA). Each sample weighing approximately 6-8 mg was placed in an aluminium pan and sealed with lids. Calibration of the equipment was carried out with indium and the samples were then subjected to an amplitude of 0.2120° C. for a period of 40 s. A heating rate of 2° C./min was employed with measurement ranging from −20° C. to 180° C. A constant nitrogen flow rate of 50 mL/min was applied during each measurement. Glass transition temperature (Tg) was found by analyzing the data collected using Trios software (TA Instruments, New Castle, USA), observing the half height of the midpoint of onset and end temperature of the samples.

### Intrinsic Dissolution Rate:

The intrinsic dissolution rate (IDR) was determined from powder compacts obtained with a hydraulic press (PerkinElmer, Hydraulische Presse Model IXB-102-9, Ueberlingen, Germany). Ball-milled powders of pure drug and spray dried powders of drug-protein mixtures were compressed into tablets. Tablets of 150 mg were directly compressed into stainless steel cylinders that served as intrinsic dissolution sample holders at a pressure of 124.9 MPa for 45 secs. Compression of tablets resulted in a flat surface of surface area 0.7854 cm² at one end of the cylinder. These cylinders were then placed in 900 ml of 0.1 M phosphate buffer (pH 7.2, 37° C.) dissolution medium and stirred using a magnetic bar at a rotation speed of 50 rpm. At predetermined time points (1, 5, 10, 15, 20, 25 and 30 min, some IDRs were only determined up to 20 min), 5 ml aliquots were withdrawn and immediately replaced with dissolution buffer. The obtained samples were then analyzed using UV spectrophotometer (see below). All dissolution experiments were conducted in triplicate.

### Ultra-Violet Spectrophotometry (UV Vis):

The concentration of each drug in the buffer was measured by an Evolution 300 UV spectrophotometer (Thermo Scientific, Cambridge, UK) at 320 nm, 272 nm, 270 nm, 265 nm and 285 nm for IND, CAR, PAR, CEL and FUR, respectively.

### Powder Dissolution (USP II Apparatus):

Powder dissolution was performed in USP type II apparatus. 200 mg of crystalline IND, amorphous IND, SD IND-WPI and physical mixture (PM) IND-WPI were added in triplicate to 50 ml phosphate buffer of pH 7.2 (sodium phosphate dibasic heptahydrate and sodium phosphate monobasic anhydrous) as the dissolution medium. The dissolution paddles were rotated at 50 rpm for 1 hour taking samples out at 1, 3, 5, 7, 10, 15, 20, 25, 30, 35, 40, 50, 60 and 120 mins. Each sample of 5 ml was taken out and replaced by dissolution medium. To separate powders from medium, the samples were filtered through a 0.45 μm syringe filter (Qmax, Frisinette ApS) and the first 2 ml was discarded to minimize losses due to adsorption. The samples were examined using UV Vis to analyze the drug concentration.

### Stability:

All samples were stored in a desiccator over silica gel (0% relative humidity) at room temperature and a physical stability study was performed for all SD samples. Each sample was analyzed by XRPD at day 0 and subsequently once every month thereafter.

### In Vivo Pharmacokinetics Studies:

The study was carried out under the study protocol approved by the Danish Animal Experiments Inspectorate (approval no. 2014-15-0201-00031). The purpose of this study was to study the performance of (i) crystalline FUR compared to (ii) amorphous FUR; (iii) physical mixture of FUR-WPI (50% FUR, 50% WPI); (iv) SD PVP-WPI (75% PVP, 25% FUR); (v) SD FUR-WPI (75% FUR, 25% WPI); (vi) SD FUR-WPI (50% FUR, 50% WPI); and (vii) SD FUR-WPI (25% FUR, 75% WPI). All ratios are in weight %. SD, XRPD, DSC and intrinsic dissolution studies were all carried using the same conditions mentioned in section 1.2.

Male Sprague Dawley Rats of 7 weeks weighing 250-348 g (Charles River, Denmark) were used for the experiments. Animals were allowed free access to water and food and were housed under controlled environmental conditions (constant temperature and humidity with a 12 h dark-light cycle). All animals were fasted for approximately 12 hours prior to being administered the drug. The rats were randomly assigned into 8 groups (each consisting of 6-8 rats) including a group receiving FUR intravenously at 1.5 mg/rat (approximately 5 mg/kg) in saline, injected in the tail vein. The remaining 7 groups were administered orally using a gavage of size 2.5 mm tablet thickness. Each tablet was a dose of 4.5 mg FUR per rat, which equals to approximately 15 mg/kg. Blood samples (0.2 ml) were collected from the tail vein after 0.25, 0.5, 1, 2, 4 and 24 hour by puncturing the tail. These blood samples were collected and stored in EDTA coated tubes and until plasma was harvested by centrifugation at 3600 g (12 min, 4° C.) and transfer into microtubes. Plasma samples were stored at −80° C. until used for further analysis. Food was given to rats after approximately 8 h after drug administration. Water was freely available for rats during the entire duration of the experiment.

Quantitative Analysis of Plasma Samples by Liquid Chromatography-Mass Spectrometry (LC-MS):

The furosemide content in plasma samples was assessed by adding 300 μl of acetonitrile to 30 μl of plasma to precipitate the proteins. An internal standard consisting of 30 μl fenofibric acid (FA) was also added to each sample. These final mixtures were then centrifuged for 10 mins at 8000 rpm (room temperature). After centrifugation, the supernatants were carefully transferred to LC-MS plates and LC-MS was performed using an Agilent technologies 1200 system with a 6140 Quadrupole detector. Chromatographic separations were carried out using an Agilent Zorbax XDB-C18 column (2.1×50 mm, 3.5 μm). The samples were eluted with a flow rate of 0.5 mL/min in a gradient mixture of 0.04% glacial acetic acid in miiliQ water (solvent A) and acetonitrile (solvent B). Each gradient program was: 0-8 min, 15% solvent B; 8-10 min, from 15% to 80% solvent B; 10-11 min, 80% solvent B; 11-11.10 min, from 80% to 15% solvent B; 11.10-14 min, 15% solvent B. The autosampler temperature was kept at 8° C. and volume of each injection sample was set to 5 μL. The LC-MS method was carried out in presence of nitrogen to assist nebulisation.

### Pharmacokinetic Analysis:

The area under the curve (AUC) of the plasma concentration with respect to time was determined by linear log trapezoidal method from time t=0 min to t=1440 min (last plasma concentration). AUC was used to calculate the absolute bioavailability (Fₐ):

$$Absolute {F}_{a} = 100 \cdot \frac{{AUC}_{P . O .}}{{AUC}_{I . V .}} \cdot \frac{{Dose}_{I . V .}}{{Dose}_{P . O .}}$$

Where P.O. stands for per oral delivery and I.V. is for intravenous dosage. The maximum FUR plasma concentration Cₘₐₓ was also determined.

### EXAMPLES

### Example 1: x-Ray Powder Diffraction of Spray Dried Drug Substance-WPI/WPH

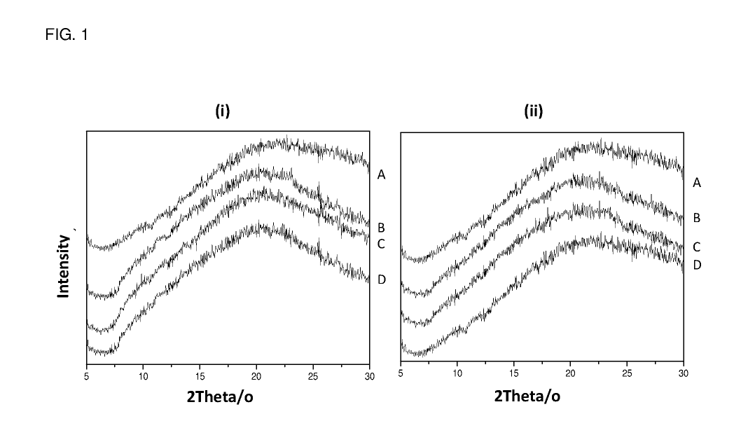

XRPD was used to analyze the amorphous (halo structure in the XRPD—no Bragg peaks in the diffractograms) or crystalline phases (distinct peaks in the diffractograms) for all samples. FIG. 1 shows the appearance of the amorphous halo in each cases proving a success in amorphization of all drug-WPI/WPH mixtures.

FIG. 10 shows the appearance of the amorphous halo in experiments where IND with rice protein isolate, soy protein isolate, egg protein isolate, collagen, gelatin, myoglobin, lysozyme and casein, respectively, were in the co-amorphous from. The figure clearly demonstrates successful amorphization in all cases. Further to this, FIG. 12 shows halo structures of IND with ovalbumin and CEL with myoglobin, lysozyme, casein, collagen and WPI, confirming the formation of co-amorphous formulations. The halo structures of CAR with myoglobin, lysozyme, casein, collagen in FIG. 18 also confirm the co-amorphous formulation.

### Example 2: Thermal Analysis of Spray Dried Drug Substance-Protein Mixtures

TGA confirmed that the residual moisture content in all amorphous drugs and the SD drug substance-protein mixtures was 3.2-8.3%. See table 1a and 1b for the detailed results.

TABLE 1a
TGA data for all amorphous drug substance and co-amorphous forms
of drug substance-WPI (including co-amorphous forms of IND with
components of WPI and co- amorphous forms of IND-WPI digested
with enzymes) and drug substance-WPH mixtures
obtained by spray drying.

| | |
|---|---|
| Powders | TGA (residual moisture content in %) |

In vitro samples

| | |
|---|---|
| A IND | 3.2 ± 0.4 |
| SD IND-WPI | 3.2 ± 0.7 |
| SD IND-WPH | 3.5 ± 0.3 |
| SD IND-α-Lactalbumin | 3.2 ± 0.2 |
| SD IND-β-Lactoglobulin | 3.6 ± 0.6 |
| SD IND-BSA | 3.7 ± 1.1 |
| SD IND-WPI (ENZ T) | 4.3 ± 0.3 |
| SD IND-WPI (ENZ T + P) | 4.2 ± 0.4 |
| SD IND-WPI (ENZ P) | 3.9 ± 0.5 |
| SD IND-WPI (ENZ P + T) | 3.9 ± 0.4 |
| A CAR | 3.1 ± 0.3 |
| SD CAR-WPI | 3.7 ± 0.2 |
| SD CAR-WPH | 3.9 ± 0.4 |
| SD PAR-WPI | 4.0 ± 0.7 |
| SD PAR-WPH | 4.1 ± 0.6 |
| A FUR | 3.6 ± 0.3 |
| SD FUR-WPI | 4.5 ± 0.3 |
| SD FUR-WPH | 4.2 ± 0.5 |

In vivo samples

| | |
|---|---|
| SD FUR-PVP (25:75) | 4.4 ± 0.8 |
| SD FUR-WPI (25:75) | 4.3 ± 0.6 |
| SD FUR-WPI (50:50) | 4.5 ± 0.3 |
| SD FUR-WPI (75:25) | 4.1 ± 0.2 |

TABLE 1b
TGA data for all co-amorphous forms of IND, CEL and CAR
with respective proteins, obtained by spray drying.

| | TGA (residual moisture |
|---|---|
| Powders | content in %) |
| SD IND-Soy | 8.1 ± 0.3 |
| SD IND-Rice | 8.3 ± 0.3 |
| SD IND-Egg | 8.1 ± 0.5 |
| SD IND-Gelatin | 1.1 ± 0.4 |
| SD IND-Collagen | 4.9 ± 0.6 |
| SD IND-Myoglobin | 6.4 ± 0.5 |
| SD IND-Lysozyme | 1.2 ± 0.3 |
| SD IND-Casein | 0.9 ± 0.2 |
| SD IND-Ovalbumin | 3.7 ± 0.2 |
| SD CEL-WPI | 2.8 ± 0.7 |
| SD CEL-Casein | 2.5 ± 0.4 |
| SD CEL-Collagen | 2.3 ± 1.2 |
| SD CEL-Lysozyme | 3.1 ± 0.8 |
| SD CEL-Myoglobin | 3.4 ± 0.7 |
| SD CAR-Casein | 3.1 ± 0.3 |
| SD CAR-Collagen | 2.4 ± 0.5 |
| SD CAR-Lysozyme | 3.6 ± 0.3 |
| SD CAR-Myoglobin | 3.1 ± 0.9 |

Each SD drug substance-protein mixture showed a single Tg (Glass transition temperature), which points that a single phase co-amorphous system has been achieved. All the co-amorphous mixtures showed increase in values Tg compared to the amorphous drug itself showing a better miscibility within the mixtures.

### Example 3: Intrinsic Dissolution Rate of Different Forms of IND

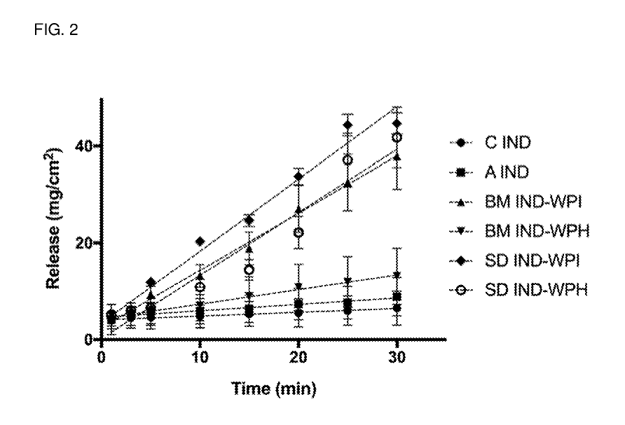

As depicted in FIG. 2, the intrinsic dissolution rate (IDR) of the amorphous ball milled IND (0.1333 mg cm⁻² min⁻¹) is 1.7 fold higher than the IDR of crystalline IND (0.0787 mg cm⁻² min⁻¹). In comparison, a substantially greater increase in the IDR was observed for the co-amorphous IND-WPI and IND-WPH mixtures. In case of spray dried IND-WPI (1.494 mg cm⁻² min⁻¹) there is a 19 fold increase in dissolution rate when compared to crystalline IND and 11 fold increase compared to ball milled amorphous IND. For spray dried IND-WPH (1.3066 mg cm⁻² min⁻¹) there is 4 fold increase from crystalline IND and about 2 fold increase from ball milled amorphous IND. There is also 1 fold increase in dissolution of spray dried IND-WPI when compared to spray dried IND-WPH. See Table 2a for the relevant line equations and intrinsic dissolution rates. Further, Table 2b presents additional line equations and intrinsic dissolution rates for co-amorphous mixtures.

TABLE 2a
Line equations of intrinsic dissolution testing of crystalline (C) and amorphous
(A) drug substances along with spray dried (SD) and ball milled (BM) co-amorphous drug
substance with whey protein isolate (WPI) and whey protein
hydrolysate (WPH), respectively.

| Sample | y (mg cm⁻² min⁻¹) | Sample | y (mg cm⁻² min⁻¹) |
|---|---|---|---|
| C IND | y = 0.0787x + 4.006 | C CAR | y = 0.0117x + 2.8082 |
| A IND | y = 0.1333x + 4.5538 | A CAR | y = 0.0214x + 2.973 |
| SD IND-WPI | y = 1.494x + 3.3209 | SD CAR-WPI | y = 0.1948x + 4.7973 |
| SD IND-WPH | y = 1.3066x + 0.1726 | SD CAR-WPH | y = 0.0794x + 3.4102 |
| BM IND-WPI | y = 1.187x + 2.4124 | BM CAR-WPI | y = 0.0266x + 3.0153 |
| BM IND-WPH | y = 0.3019x + 4.2494 | BM CAR-WPH | y = 0.0307x + 2.979 |
| WPI | y = 3.8317x + 3.8477 | C PAR | y = 0.1632x + 2.3462 |
| WPH | y = 3.2347x + 42.311 | A PAR | y = 0.201x + 4.5947 |
| SD IND-α lactalbumin | y = 1.1065x + | SD PAR-WPI | y = 0.5433x + 3.273 |
| | 5.1568 | | |
| SD IND-β lactoglobulin | y = 0.3468x + 5.311 | SD PAR-WPH | y = 0.4664x + 2.4197 |
| SD IND-BSA | y = 0.2861x + 5.4 | BM PAR-WPI | y = 0.3386x + 3.0738 |
| SD IND-WPI (ENZ T) | y = 0.2512x + | BM PAR-WPH | y = 0.2865x + 3.4489 |
| | 4.4218 | | |
| SD IND-WPI (ENZ T + P) | y = 0.2271x + 7.1504 | C FUR | y = 0.1024x + 2.3685 |
| SD IND-WPI (ENZ P) | y = 0.1131x + 4.6117 | A FUR | y = 0.1548x + 2.4064 |
| SD IND-WPI (ENZ P + T) | y = 1.1917x + 7.4698 | SD FUR-WPI | y = 0.4115x + 5.0747 |
| | | SD FUR-WPH | y = 0.2359x + 4.3598 |
| | | BM FUR-WPI | y = 0.2279x + 2.1756 |
| | | BM FUR-WPH | y = 0.1972x + 2.234 |

TABLE 2b
Line equations for intrinsic dissolution testing performed on all
co-amorphous IND-protein, CEL-protein and CAR-protein
mixtures. All mixtures were prepared by spray drying (SD).

| Sample | y (mg cm⁻² min⁻¹) |
|---|---|
| SD IND-Gelatin | y = 0.6263x + 3.5063 |
| SD IND-Egg | y = 0.3502x + 2.2251 |
| SD IND-Rice | y = 0.1952x + 2.5039 |
| SD IND-Soy | y = 0.1801x + 2.6814 |
| SD IND-Lysozyme | y = 1.0676x + 4.2697 |
| SD IND-Myoglobin | y = 0.9113x + 2.4847 |
| SD IND-Collagen | y = 0.2924x + 2.5942 |
| SD IND-Casein | y = 0.2224x + 2.2982 |
| SD IND-Ovalbumin | y = 0.2733x + 2.8722 |
| SD CEL-WPI | y = 0.9972x + 4.7968 |
| SD CEL-Casein | y = 0.9714x + 2.9813 |
| SD CEL-Collagen | y = 0.6629x + 1.8036 |
| SD CEL-Lysozyme | y = 0.2019x + 1.0872 |
| SD CEL-Myoglobin | y = 0.2628x + 1.8702 |
| SD CAR-Casein | y = 0.1925x + 4.0547 |
| SD CAR-Collagen | y = 0.1694x + 3.5951 |
| SD CAR-Lysozyme | y = 0.0737x + 3.0801 |
| SD CAR-Myoglobin | y = 0.092x + 3.3139 |

FIGS. 11 (i) and (ii) show the intrinsic dissolution rate (IDR) for the co-amorphous forms of IND with various proteins, where the co-amorphous forms are prepared by spray-drying. Spray dried IND-WPI (1.494 mg cm⁻² min⁻¹) has a 19 fold increase in dissolution rate when compared to crystalline IND and an 11 fold increase compared to ball milled amorphous IND. For spray dried IND-WPH (1.3066 mg cm⁻² min⁻¹) there is 4 fold increase from crystalline IND and about 2 fold increase from ball milled amorphous IND. There is also a 1 fold increase in the dissolution rate of spray dried IND-WPI when compared to spray dried IND-WPH. The dissolution rate of SD IND-OVALBUMIN, SD IND-GELATIN, SD IND LYSOZYME, SD IND-MYOGLOBIN, SD IND-COLLAGEN and SD IND-CASEIN are 3.5, 7.9, 13.5, 11.6, 3.7, 2.8 fold higher than C IND and 2, 4.7, 8, 6.8, 2.2, 1.7 fold higher than A IND, respectively. On the other hand SD IND-EGG, SD IND-RICE and SD IND-SOY are 4.5, 2.5, 2.3 fold higher than C IND and 2.6, 1.5, 1.4 fold higher than A IND.

Proteins were paired with the acidic drug IND (pKa: 4.5), neutral drug CEL (pKa: 11.1) and basic drug CAR (pKa: 7.8) based on their isoionic points (pI), pH value at which a zwitterion molecule has an equal number of positive and negative charges, and subsequently the intrinsic dissolution rate (IDR) was determined (FIG. 13 (i), 13 (ii), 13 (iii)). The pI's of lysozyme, myoglobin, collagen and casein are 10.7, 7.4, 5.8 and 4.6, respectively. The pI of WPI is ˜5, since WPI is a mixture of α-lactalbumin, ρ-lactoglobulin and BSA, which have pI's of 5.0, 5.2 and 5.2, respectively. For co-amorphous mixtures with IND, SD IND-LYSOZYME (1.0676 mg cm⁻² min⁻¹) had the highest IDR followed by SD IND-MYOGLOBIN (0.9113 mg cm⁻² min⁻¹), SD IND-COLLAGEN (0.2924 mg cm⁻² min⁻¹) and SD IND-CASEIN (0.2224 mg cm⁻² min⁻¹). This shows that at pH 7.2 the negatively charged IND achieves a higher IDR when paired with a co-former protein with high pI, indicating that electrostatic attraction between the negatively charged IND and the protein with net positive charge has a positive influence on the dissolution rate.

A similar pattern was found in case of CAR, which is positively charged at pH 7.2 and showed higher IDR with decreasing pI of proteins used to form the co-amorphous mixtures. SD CAR-CASEIN (0.1925 mg cm⁻² min⁻¹), SD CAR-COLLAGEN (0.1694 mg cm⁻² min⁻¹) and SD CAR-WPI (0.1948 mg cm⁻² min⁻¹) showed higher IDR compared to CAR mixed with proteins with a net positive charge, lysozyme and myoglobin. SD CAR-CASEIN was 2.6 fold higher than SD CAR-LYSOZYME (0.0737 mg cm⁻² min⁻¹) and 2.1 fold higher than SD CAR-MYOGLOBIN (0.092 mg cm⁻² min⁻¹), whereas SD CAR-COLLAGEN was 2.3 and 1.8 fold higher than SD CAR-LYSOZYME and SD CAR-MYOGLOBIN, respectively. This suggests that electrostatic attraction between the drug molecule and co-former protein has a positive influence on the resulting IDR compared with electrostatic repulsion. Interestingly, CEL, which is neutral at pH 7.2 also showed higher IDR when combined with proteins with a net negative charge as co-former. SD CEL-CASEIN (0.9714 mg cm⁻² min⁻¹) showed the highest IDR followed by SD CEL-COLLAGEN (0.6629 mg cm⁻² min⁻¹), SD CEL-MYOGLOBIN (0.2628 mg cm⁻² min⁻¹) and SD CEL-LYSOZYME (0.2019 mg cm⁻² min⁻¹). This may be due to neutral charge at pH 7.2 and other additional properties of CEL.

In all cases, when using WPI as a co-former the IDR was found to be higher than that observed with other proteins, irrespective of the pI and independent of the nature of the drug (acidic, basic or neutral). For further visualization of the correlation between the IDR of drugs with different charge and proteins with different net charge the IDR of the different drug-protein combinations were plotted against the pI of the proteins (FIG. 15i-iii). There is a good correlation between the pI of the proteins and the resulting IDR of the co-amorphous mixtures except for WPI when used as a co-former with IND. This may be explained by the composition and properties of WPI. As mentioned, WPI consists of a mixture of multiple proteins, which could result in higher heterogeneity of the resulting co-amorphous mixtures compared with a co-amorphous mixture consisting of a single protein and drug. This could have a positive effect on the dissolution rate of the drug. Further, it is believed that certain properties of the proteins of WPI make them especially suitable for forming stable interactions with drug molecules, which result in enhanced dissolution rate of the drug.

FIG. 14 (i) shows co-amorphous forms of IND spray dried with WPI and with other proteins that represent mixtures of several proteins together. Egg protein isolate (EGG) is a mixture consisting mainly of ovalbumin, ovomucoid, ovomucin and lysozyme, whereas, rice protein isolates (RICE) consist of glutenin, globulin, albumin and prolamin. On the other hand, soy protein isolates (SOY) are mixture of globular proteins, conglycinin and glycinin, and gelatin is essentially denatured and hydrolyzed collagen. The IDR of SD IND-EGG was found to be 1.9 fold higher than that of SD IND-SOY and 1.8 fold higher than that of SD IND-RICE. Furthermore, SD IND-gelatin showed a 1.8 fold higher intrinsic dissolution than SD IND-EGG. From all IND-protein mixtures, again SD IND-WPI showed the highest intrinsic dissolution, which was 2.4 fold higher than SD IND-gelatin. Overall, SD IND-WPI had the highest dissolution rate followed by SD IND-gelatin, SD IND-EGG, SD IND-RICE and SD IND-SOY. FIG. 17 shows that the isoionic points of these proteins (pI) did not have a direct co-relation with the IDR observed, most likely due to the proteins being a combination of several other proteins. SD IND-WPI, however, had the highest dissolution rate. Each of the proteins comprising WPI indicated a relatively high IDR, especially α-lactalbumin, when used alone (Table 2a) with the drug molecule and resulted in an even higher IDR when mixed as WPI. SD IND-gelatin also indicated a high dissolution rate compared with many other proteins but but both SD IND-WPI and SD IND-WPH resulted in a higher dissolution rate.

FIG. 14 (ii) shows SD IND-protein co-amorphous mixtures where the co-former proteins were selected based on their molecular weight (Mw), BSA having the highest Mw (≈66500), followed by ovalbumin (≈45000), casein (≈23000) and WPI (≈15000). These four proteins, BSA, ovalbumin, casein and WPI also have similar pI's of 5.2, 4.8, 4.6 and ˜5, respectively. Interestingly it was found that the dissolution rate of SD IND-BSA was around 1.05 fold higher than that of SD IND-OVALBUMIN and 1.3 fold higher than SD IND-CASEIN, although they were relatively similar. This may suggest that the Mw of the proteins has a slight influence on the resulting dissolution rate of the co-amorphous mixture, possibly due to high diversity in interaction formed with high Mw proteins. FIG. 16 (i) also illustrates this trend. Here, SD IND-WPI was again an outlier, resulting in higher IDR irrespective of its lower Mw.

### Example 3: Intrinsic Dissolution Rate of Different Forms of CAR, PAR and FUR

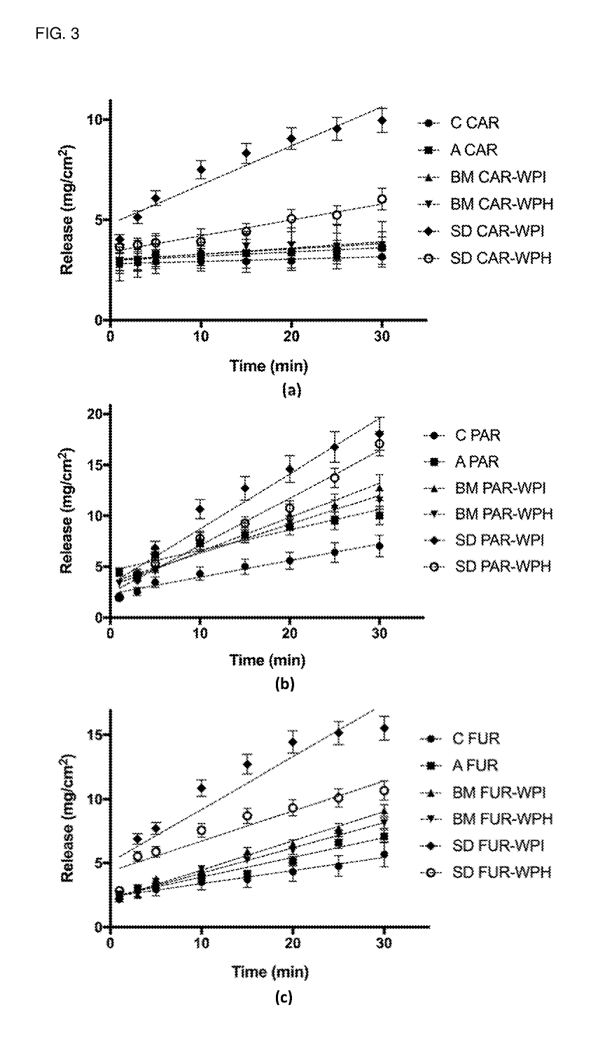

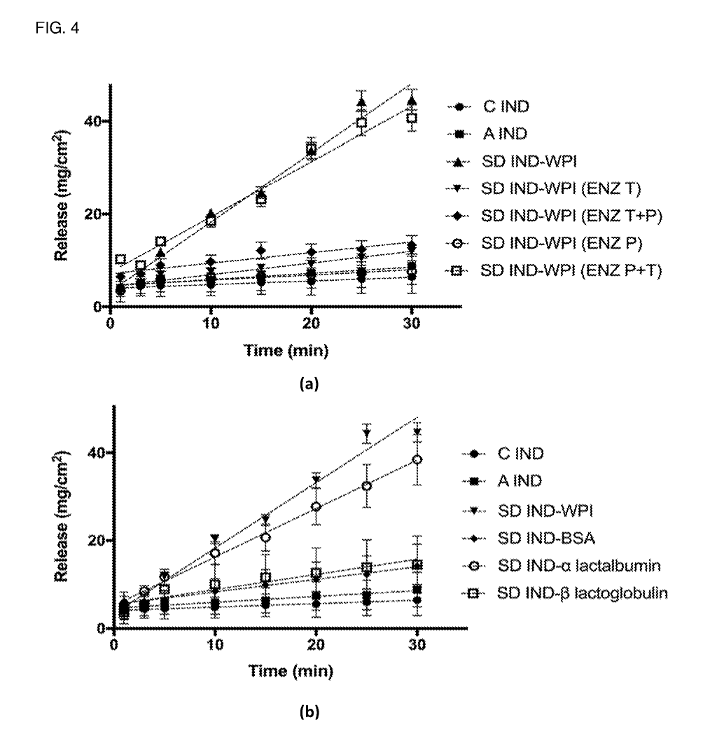

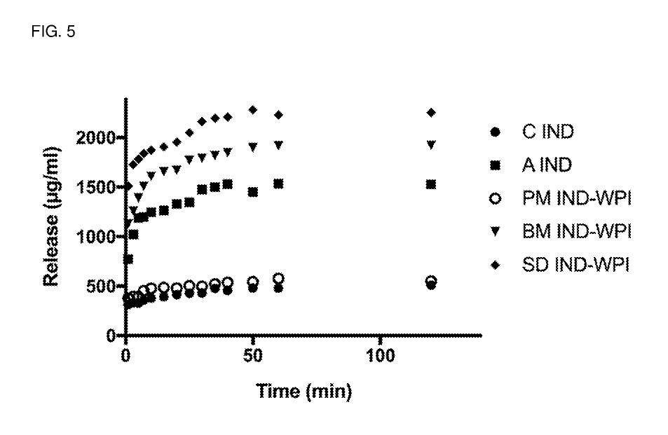

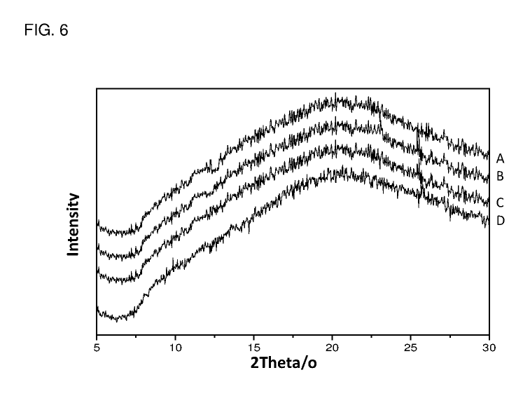

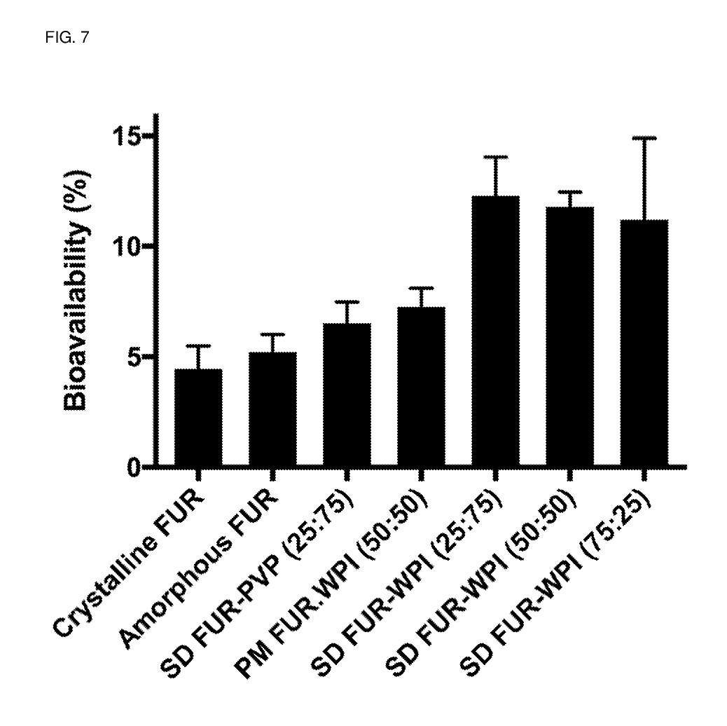

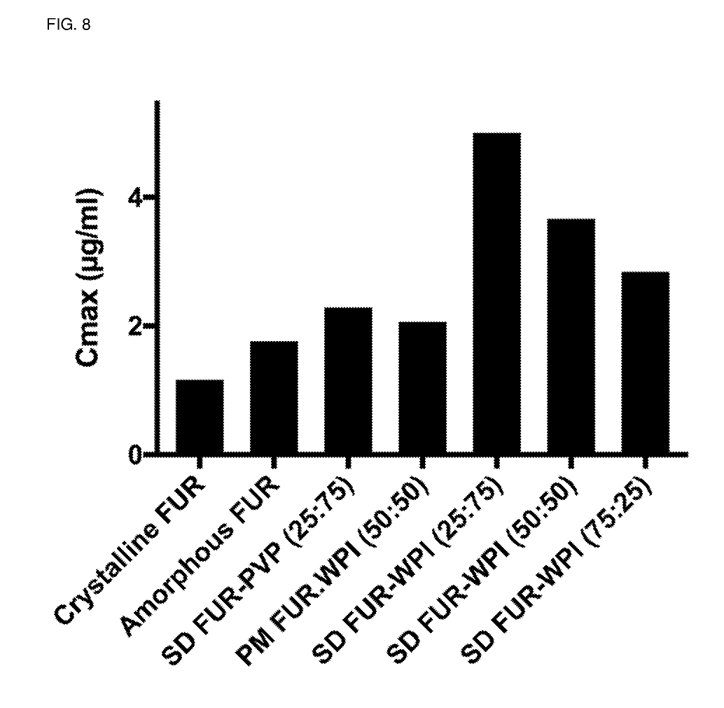

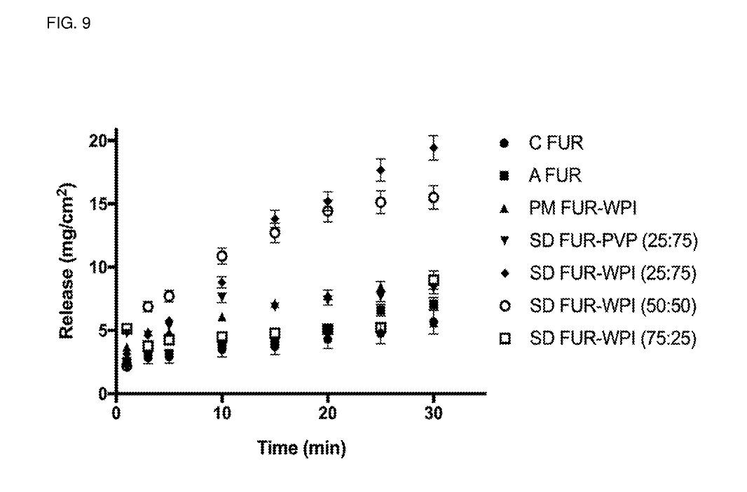

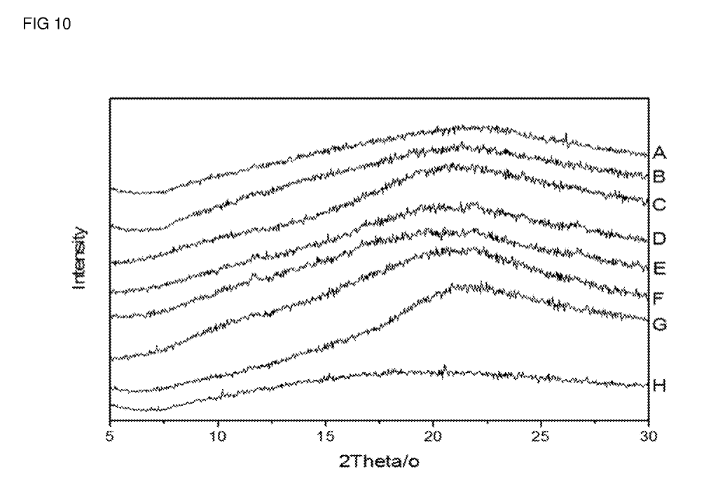

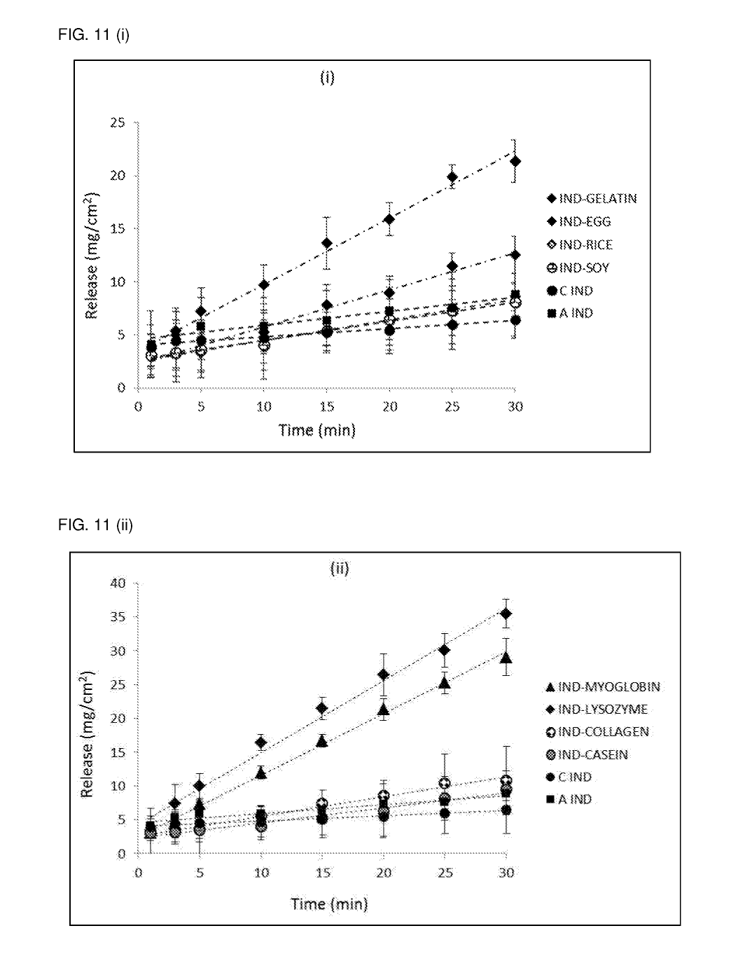

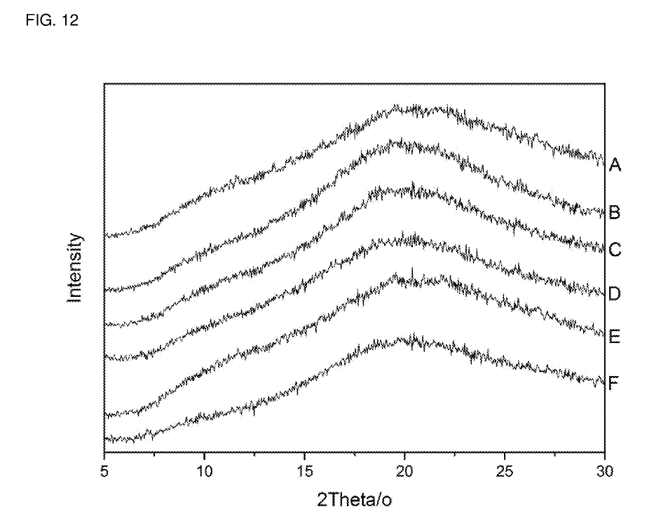

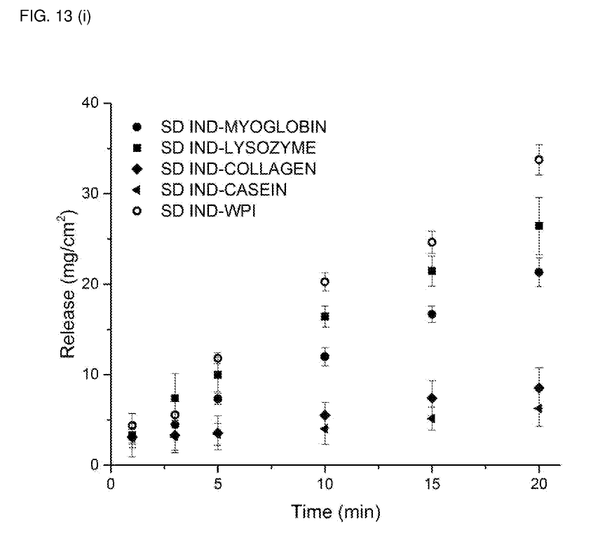

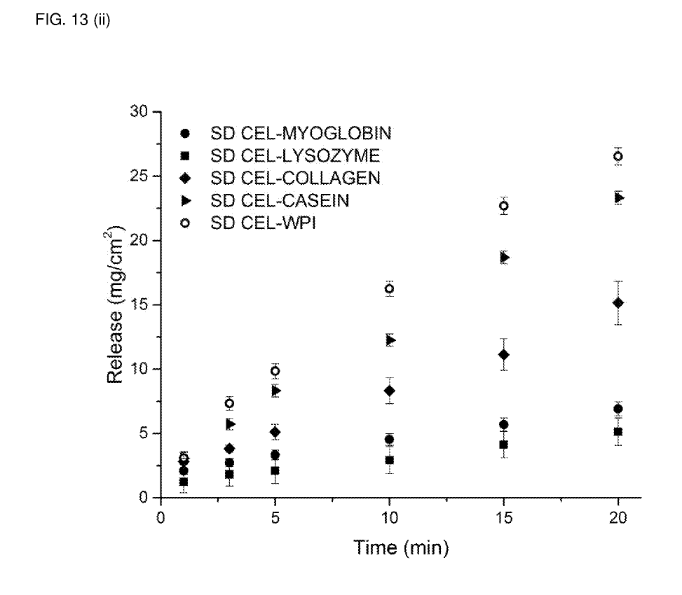

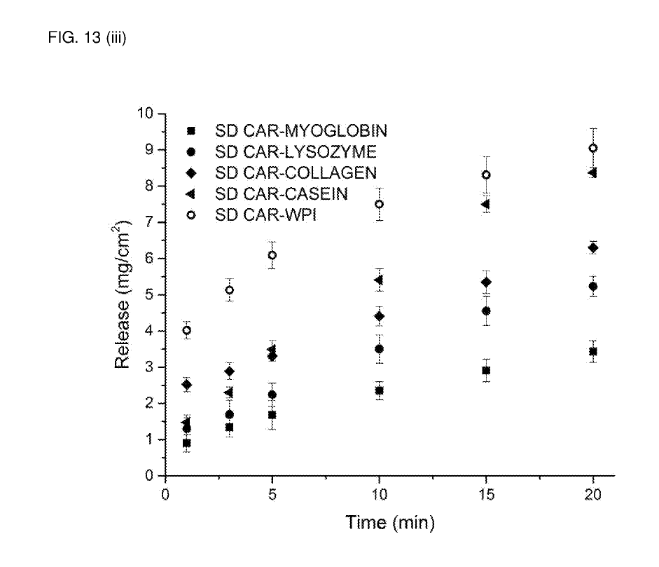

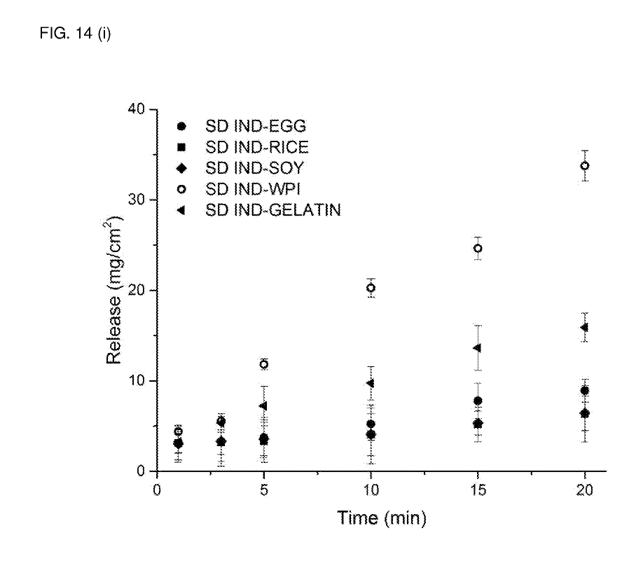

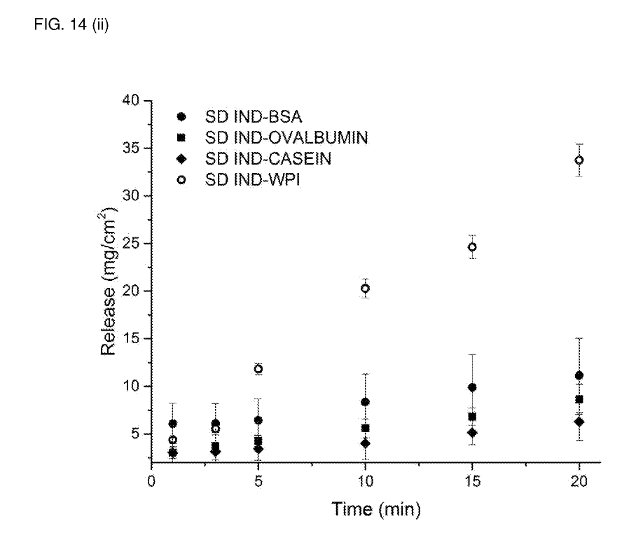

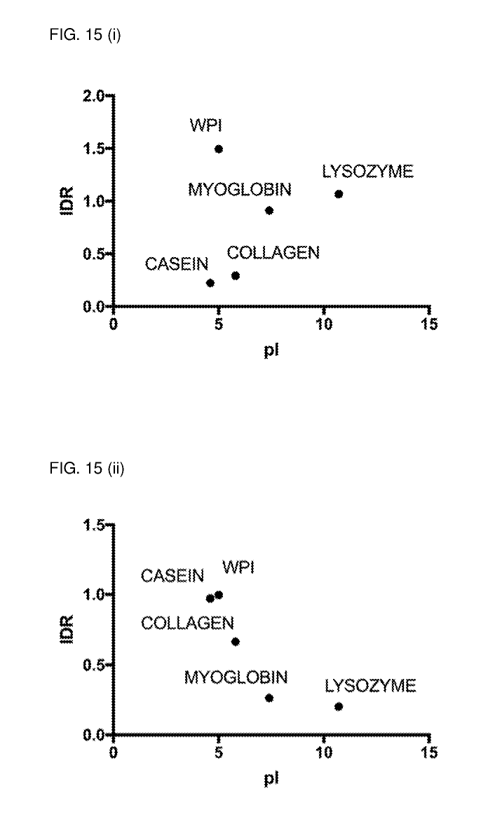

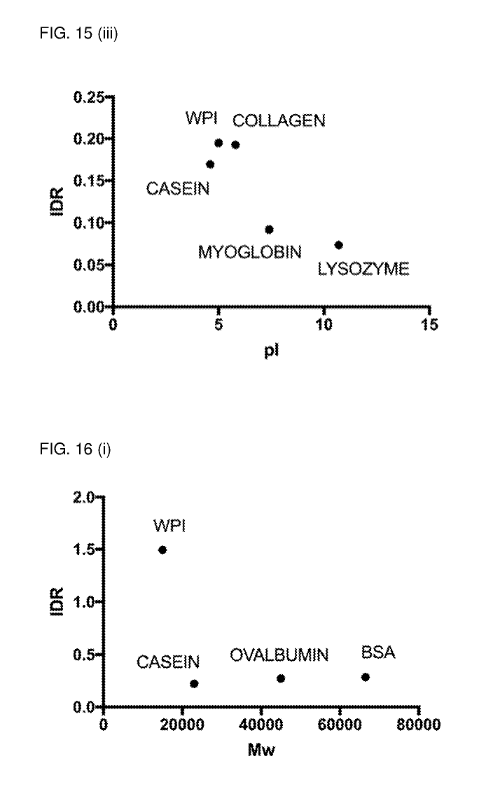

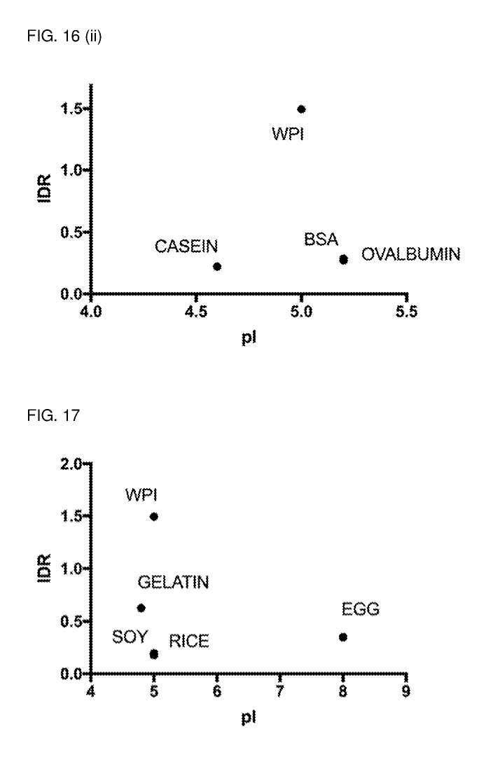

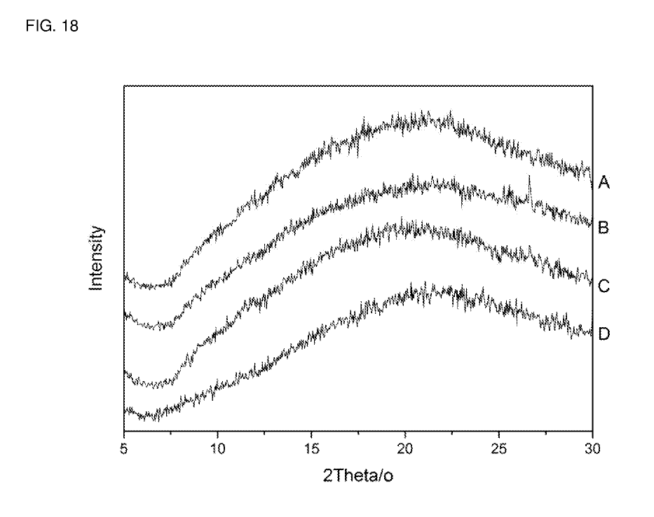

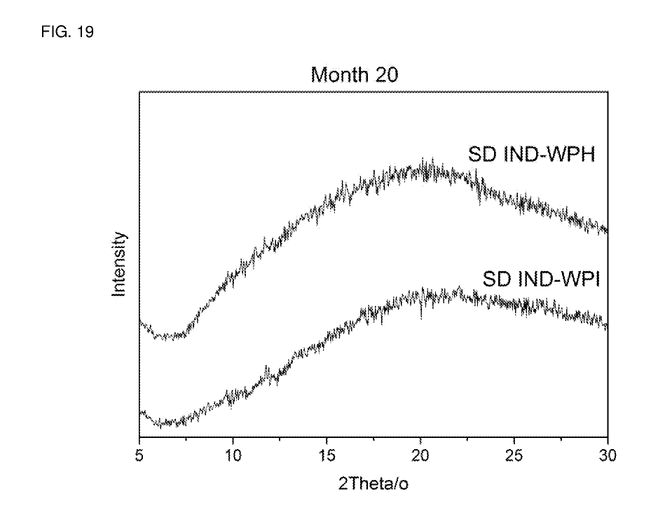

FIG. 3 depicts the IDR of different forms of CAR (FIG. 3A), PAR (FIG. 3B), and FUR (FIG. 3C). See Table 2 for the relevant line equations.

FIG. 3 demonstrates that the IDR of ball milled amorphous CAR (0.0214 mg cm⁻² min⁻¹), PAR (0.201 mg cm⁻² min⁻¹) and FUR (0.514 mg cm⁻² min⁻¹) is 1.8, 1.2, and 1.5 fold higher than the IDR of crystalline CAR (0.0117 mg cm⁻² min⁻¹), PAR (0.1632 mg cm⁻² min⁻¹) and FUR (0.1024 mg cm⁻¹ min⁻¹) respectively.

Moreover, there is a great increase in the IDR of for the co-amorphous drug substance-WPI/WPH mixtures. The IDR of the spray dried (SD) CAR-WPI and SD CAR-WPH (0.194 mg cm⁻² min⁻¹ and 0.0794 mg cm⁻² min⁻¹, respectively) shows nearly a 17 (WPI) and a 7 (WPH) fold increase compared to crystalline CAR and a 9 (WPI) and 3.7 (WPH) fold increase compared to ball milled amorphous CAR.

In case of SD PAR-WPI and SD PAR-WPH (0.5433 mg cm⁻¹ min⁻¹ and 0.4664 mg cm⁻¹ min⁻¹) there is a 3.3 (WPI) and 2.8 (WPH) fold increase in dissolution rate when compared to crystalline PAR and 2.7 (WPI) and 2.3 (WPH) fold increase compared to the individual ball milled amorphous PAR.

For SD FUR-WPI and SD FUR-WPH (0.4115 mg cm⁻² min⁻¹ and 0.2359 mg cm⁻¹ min⁻¹) a 4 (WPI), 2.3 (WPH) fold increase was observed in dissolution rate when compared to crystalline FUR and 2.6 (WPI), 1.5 (WPH) fold increase compared to the individual amorphous (BM) FUR. It can be concluded from FIGS. 2 and 3 that spray dried drug-WPI mixtures has the highest dissolution rate when compared to its crystalline or amorphous counterparts.

### Example 4: Intrinsic Dissolution Rate of Amorphous IND with Different WPI Components

FIG. 4 shows that the IDR of WPI (3.8317 mg cm⁻² min⁻¹) is 3.4, 11 and 13.4 folds higher than its components: α-lactalbumin, β-lactoglobulin and BSA, respectively. SD IND-WPI is 6 fold higher than SD IND-WPI with trypsin, 13.2 fold than SD IND-WPI with pepsin and 6.6 fold more than SD IND-WPI with trypsin+pepsin (trypsin added first). SD IND-WPI is 1.25 fold higher SD IND-WPI with pepsin+trypsin (pepsin added first). Consequently, it can be concluded that the intact native form of WPI provides with the highest dissolution rate when compared to co-amorphous forms digested with enzymes.

### Example 5: Powder Dissolution Studies

As seen in FIG. 5, we can see that co-amorphous SD IND-WPI shows higher dissolution rate compared to BM IND-WPI. Amorphous IND itself shows more dissolution than the PM IND-WPI. The solubility of IND in its amorphous state is more than double the value crystalline IND. This is due to the solubility of a compound in the amorphous form is higher than in the more stable crystalline form because the Gibbs free energy is higher. This increase in dissolution rate from the amorphous drug alone is due to the increase in molecular interaction upon co-amorphisation.

### Example 6: Stability Studies

FIG. 6 depicts the physical stability of co-amorphous forms of WPI and WPH with IND, CAR, FUR, and PAR, respectively. Amorphous IND, CAR, FUR and PAR were found to be stable for less than a week shown by recrystallization from XRPD. In contrast, the co-amorphous spray dried drug substance-protein mixtures were found to be stable for several months. SD IND-WPI and SD IND-WPH were found to be stable for more than 20 months (FIG. 19) whereas most other SD IND-protein co-amorphous forms such as for example SD IND-gelatin, SD IND-BSA and SD IND-collagen (see Table 3 below for detailed stability study) were only stable for 2-3 months. Co-amorphous formulations of WPI and WPH with the drugs CAR and FUR were also stable up to 8 and 18 months, respectively. The co-amorphous form of SD CEL-WPI was also stable for more than 8 months. Hence, WPI and WPH were the proteins and co-formers for co-amorphous mixtures with the best stabilizing properties for all of the investigated drugs. It was also more stable than a solid dispersions prepared using PVP (a commonly used co-former for amorphous formulations) and drug, even at a higher drug concentration (drug loading). This indicates that WPI and WPH are not only performing superiorly compared with other proteins and protein mixtures with regards to dissolution when combined with poorly soluble drugs to form co-amorphous mixtures or solid dispersions. They are also performing superiorly compared with other proteins and protein mixtures with regards to physical stability with several fold increase in stability observed for WPI and WPH.

TABLE 3
Stability data showing the number of months SD drug-protein
mixtures remained co-amorphous. XRPD was used to conclude
this data. On recrystallization, mixtures showed the peaks of
respective drugs in diffractograms. The stability study was stopped
only for the samples which showed crystalline peaks.

| | Number of months at | Number of months at |
|---|---|---|
| | which recrystallization | which drug was still |
| SD formulations | of drug was observed | amorphous |
| IND-WPI | — | 20 |
| IND-WPH | — | 20 |
| SD IND-α lactalbumin | 3 | 2 |
| SD IND-β lactalbumin | 3 | 2 |
| IND-BSA | 3 | 2 |
| IND-GELATIN | 2 | 1 |
| IND-EGG | 3 | 2 |
| IND-WPI (ENZ P) | 2 | 1 |
| IND-WPI (ENZ T + P) | 2 | 1 |
| IND-WPI (ENZ P) | 2 | 1 |
| IND-WPI (ENZ P + T) | 2 | 1 |
| IND-RICE | 2 | 1 |
| IND-SOY | 2 | 1 |
| IND-LYSOZYME | 3 | 2 |
| IND-MYOGLOBIN | 3 | 2 |
| IND-COLLAGEN | 2 | 1 |
| IND-CASEIN | 2 | 1 |
| CAR-WPI | 8 | 7 |
| CAR-WPH | 8 | 7 |
| PAR-WPI | 2 | 1 |
| PAR-WPH | 2 | 1 |
| FUR-WPI | 18 | 17 |
| FUR-WPH | 18 | 17 |
| PVP 75%-FUR 25% | 3 | 2 |
| WPI 75%-FUR 25% | 18 | 17 |
| WPI 50%-FUR 50% | 16 | 15 |
| WPI 25%-FUR 75% | 5 | 4 |
| IND-OVALBUMIN | 6 | 5 |
| CEL-CASEIN | 6 | 5 |
| CEL-COLLAGEN | 6 | 5 |
| CEL-LYSOZYME | 4 | 3 |
| CEL-MYGLOBIN | 4 | 3 |
| CEL-WPI | — | 8 |
| CAR-CASEIN | — | 1 |
| CAR-COLLAGEN | — | 1 |
| CAR-LYSOZYME | — | 1 |
| CAR-MYGLOBIN | — | 1 |

### Example 7: In Vivo Studies

FIG. 7 depicts the bioavailability of co-amorphous (spray dried) forms of FUR and WPI following oral administration to rats. The SD WPI:FUR (75% WPI, 25% FUR) showed the highest bioavailability (11.4%) followed closely by SD WPI:FUR (50% WPI, 50% FUR) (11.3%) and SD WPI:FUR (25% WPI, 75% FUR) (10.6%). This indicates that the bioavailability increases with increasing WPI content. The bioavailability of SD WPI:FUR samples was significantly higher than that of SD PVP:FUR (6.3%) and the physical mixture. Crystalline FUR showed the lowest bioavailability (4.7%) as expected and was followed by amorphous FUR (5.1%), both of which were significantly lower than the SD WPI:FUR samples. The ratios of WPI and FUR were varied by changing the content of WPI while the content of FUR is kept constant.

FIG. 8 depicts the maximum concentration (Cmax) following oral administration to rats. The pattern of Cmax values was in line with the bioavailability results. An increase in the amount of WPI in the co-amorphous resulted in increased Cmax levels.

### Example 8: Intrinsic Dissolution Rate of Compounds Used for In Vivo Experiments

FIG. 9 depicts the IDR of the compositions used for the in vivo experiments. The IDR of the SD WPI-FUR (75% WPI, 25% FUR) was found to have the highest dissolution rate. It was 5.67 fold higher than the crystalline FUR and 3.7 fold higher than the amorphous FUR. It was followed by SD WPI-FUR (50% WPI, 50% FUR) which was 4 fold more than crystalline and 2.6 fold more than amorphous FUR. Interestingly, it was found that traditionally used SD PVP/FUR (75% PVP, 25% FUR) was only 1 fold higher than the SD WPI-FUR (25% WPI, 75% FUR). Amorphous FUR was 0.69 fold higher than PM WPI-FUR (50% WPI, 50% FUR) See table 4 for the relevant line equations.

TABLE 4
Line equations for intrinsic dissolution testing for all mixtures
used in in vivo studies.

| Sample | y (mg cm⁻¹min⁻¹) |
|---|---|
| C FUR | y = 0.1024x + 2.3685 |
| A FUR | y = 0.1548x + 2.4064 |
| PM FUR-WPI (50% PUR, 50% WPI) | y = 0.1065x + 4.6238 |
| SD PVP 75%-FUR 25% | y = 0.1213x + 4.9021 |
| SD WPI 75%-FUR 25% | y = 0.5811x + 3.1377 |
| SD WPI 50%-FUR 50% | y = 0.4114x + 5.0757 |
| SD WPI 25%-FUR 75% | y = 0.119x + 3.7316 |

C: crystalline,
A: amorphous,
PM: physical mixture,
SD: spray dried.

### OVERALL CONCLUSION

From the above examples, we can conclude that various proteins, notably WPI, are promising new excipients for the co-amorphization of crystalline drug substance. Co-amorphous forms of the drugs IND, CAR, FUR, PAR and CEL show remarkably higher dissolution rate compared to the crystalline or mono-amorphous forms of the drug substances, but most notably also to other competing technologies that are developed to improve dissolution rate and solubility of poorly soluble drug substances. Improved bioavailability and PK-profile was also observed for all formulations with WPI, compared with mono-amorphous drug substances and solid dispersions (with PVP) and physical mixtures. Furthermore, the co-amorphous drug substance-WPI forms also showed an increased physical stability compared to their mono-amorphous counterparts.

1. A co-amorphous form of a drug substance and a protein,
wherein the protein is selected from whey protein isolate, whey protein hydrolysate, soy protein isolate, soy protein hydrolysate, myoglobin, lysozyme, egg protein isolate, egg white protein isolate, egg white protein hydrolysate, egg protein isolate, ovalbumin, casein, alpha-lactalbumin, beta-lactoglobulin, immunoglobulin G, rice protein isolate, rice protein hydrolysate, or collagen, or mixtures thereof.

2-25. (canceled)

26. The co-amorphous form according to claim 1, wherein the protein is selected from whey protein isolate, whey protein hydrolysate, alpha-lactalbumin, beta-lactoglobulin, immunoglobulin G, or lactoferrin, or mixtures thereof.

27. The co-amorphous form according to claim 1, wherein the protein is whey protein isolate and/or whey protein hydrolysate.

28. The co-amorphous form according to claim 1, wherein the protein comprises beta-lactoglobulin, alpha-lactalbumin, immunoglobulin G, bovine serum albumin, or lactoferrin.

29. The co-amorphous form according to claim 1, wherein the protein comprises:
i) at least about 50 w/w of beta-lactoglobulin,
ii) at least about 10% w/w of alpha-lactalbumin,
iii) at least about 10% w/w of immunoglobulin G,
iv) at the most about 10% w/w of bovine serum albumin, or
v) at least about 1% w/w of lactoferrin, or
vi) mixtures thereof.

30. The co-amorphous form according to claim 1, wherein the protein comprises:
i) from about 50 to about 70% w/w of beta-lactoglobulin,
ii) from about 10 to about 25% w/w of alpha-lactalbumin,
iii) from about 10 to about 20% w/w of immunoglobulin G,
iv) from about 1 to about 10% w/w of bovine serum albumin,
v) from about 0 to about 10% w/w of lactoferrin,
vi) and from about 0 to 5% w/w of other proteins, peptides, carbohydrates, lipids, minerals, vitamins or water.

31. The co-amorphous form according to claim 1, wherein the protein comprises:
i) from about 55 to about 65% w/w of beta-lactoglobulin,
ii) from about 15 to about 21% w/w of alpha-lactalbumin,
iii) from about 13 to about 14% w/w of immunoglobulin G,
iv) about 7% w/w of bovine serum albumin, and
v) from about 0 to about 3% w/w of lactoferrin.

32. The co-amorphous form according to claim 1, wherein the protein comprises:
i) about 65% w/w of beta-lactoglobulin,
ii) about 15% w/w of alpha-lactalbumin,
iii) about 13% w/w of immunoglobulin G,
iv) about 7% w/w of bovine serum albumin, or
v) about 0% w/w of lactoferrin.

33. The co-amorphous form according to claim 1, wherein the protein comprises:
i) about 55% w/w of beta-lactoglobulin,
ii) about 21% w/w of alpha-lactalbumin,
iii) about 14% w/w of immunoglobulin G,
iv) about 7% w/w of bovine serum albumin, and
v) about 3% w/w of lactoferrin.

34. The co-amorphous form according to claim 1, wherein the co-amorphous form comprises from 10 to 90% w/w of the drug substance and from 10 to 90% w/w of the protein.

35. The co-amorphous form according to claim 1, wherein the co-amorphous form comprises from 25 to 75% w/w of the drug substance and from 25 to 75% w/w of the protein.

36. A method of using the co-amorphous form according to claim 1 in a therapy for a subject comprising administering the co-amorphous form according to claim 1 to a subject in need thereof.

37. A method using a cosmetic comprising applying the co-amorphous form according to claim 1 to a subject.

38. A method for preparing a composition comprising spray drying, solvent evaporating, freeze drying, precipitation from a supercritical fluid, melt quenching, hot melt extrusion, electrospinning, 2D printing, 3D printing, or milling the co-amorphous form according to claim 1.

39. A method for preparing a co-amorphous form according to claim 1 comprising:
i) placing a drug substance and a protein in a container, and sealing the container,
ii) physically disordering the drug substance together with the protein by mechanical activation until the drug substance and the protein are completely disrupted resulting in a co-amorphous product, and
iii) simultaneously mixing of the substance and the protein to obtain a homogeneous co-amorphous one-phase system comprising the drug substance and the protein.

40. A method for preparing a co-amorphous form as defined in claim 1 comprising:
i) dissolving a drug substance and a protein in a solvent or solvent mixture to form a single phase solution, and
ii) removing the solvent from the resulting solution from step i) to obtain a homogeneous one-phase co-amorphous mixture comprising the drug substance and the protein.

41. A method for preparing a co-amorphous form as defined in claim 1 comprising:
i) dissolving a drug substance and a protein in a solvent or solvent mixture to form a single phase solution,
ii) freezing the single phase solution from step i), and
iii) removing the solvent or solvent mixture through sublimation from the resulting frozen single phase from step ii) to obtain a homogeneous one-phase co-amorphous mixture comprising the drug substance and the protein.

42. A method according to claim 40, wherein the solvent is water.

43. A method for preparing a co-amorphous form as defined in claim 1 comprising:
i) mixing a substance and a protein to obtain a physical mixture of both components,
ii) disordering the resulting physical mixture from step i) by heating the mixture above the melting point of either the substance, the protein or both together to obtain a homogeneous single phase melt comprising both substance and protein, and
iii) cooling of the single phase melt from step ii) to below the glass transition temperature to obtain a homogeneous one-phase co-amorphous mixture comprising the substance and the protein.

44. The co-amorphous form of claim 1 further comprising at least one pharmaceutically, cosmetically or veterinary acceptable excipient.

45. The co-amorphous form according to claim 1, wherein the co-amorphous form has a stability of at least 5 weeks or more when stored in a desiccator over silica gel at 0% relative humidity and room temperature of 18-25° C. and analyzed by XRPD.

46. The co-amorphous form according to claim 45, wherein the co-amorphous form has a stability of 8 weeks or more.

47. The co-amorphous form according to claim 45, wherein the co-amorphous form has a stability of 15 weeks or more.

48. The co-amorphous form according to claim 1, wherein an increase in intrinsic dissolution rate of the co-amorphous form is at least 2 fold higher than the dissolution rate of the crystalline drug substance.

49. The co-amorphous form according to claim 48, wherein the increase in intrinsic dissolution rate of the co-amorphous form is at least 5 fold higher than the dissolution rate of the crystalline drug substance.
